(19) United States
(12) Patent Application Publication
(10) Pub. No.: US 2022/0176036 A1
(43) Pub. Date: Jun. 9, 2022

(54) PUMP CASSETTE WITH RADIO FREQUENCY IDENTIFICATION TAG

(71) Applicant: ICU Medical, Inc., San Clemente, CA (US)

(72) Inventors: Marshall E. Fryman, Libertyville, IL (US); Matteo D. Picinich, Temecula, CA (US)

(21) Appl. No.: 17/457,324

(22) Filed: Dec. 2, 2021

Related U.S. Application Data
(60) Provisional application No. 63/121,360, filed on Dec. 4, 2020.

(51) Int. Cl. A61M 5/168; A61M 5/36; A61M 5/14; G06K 19/07; G06K 7/10

(52) U.S. Cl. CPC A61M 5/16813; A61M 5/36; A61M 5/1409; G06K 19/0723; A61M 2205/50; A61M 2205/6054; A61M 2205/3584; A61M 2205/52; A61M 2205/276; G06K 7/10366

(57) ABSTRACT

Aspects of this disclosure relate to a pump cassette with a radio frequency identification (RFID) tag. The RFID tag can store information associated with the pump cassette, such as usage information and/or a unique identifier. The RFID tag can be embedded in a component of the pump cassette in certain embodiments. Related infusion pumps, infusion sets, and infusion systems are disclosed.

### CROSS REFERENCE TO PRIORITY APPLICATION

Any and all applications for which a foreign or domestic priority claim is identified in the Application Data Sheet as filed with the present application are hereby incorporated by reference under 37 C.F.R. § 1.57. This application claims the benefit of priority of U.S. Provisional Application No. 63/121,360, filed Dec. 4, 2020 and titled “PUMP CASSETTE WITH RADIO FREQUENCY IDENTIFICATION TAG AND/OR TUBING SET WITH LIGHT SET,” the disclosure of which is hereby incorporated by reference in its entirety and for all purposes.

### BACKGROUND

### Technical Field

Embodiments of this disclosure relate the field of infusion systems and/or components thereof.

### Description of the Related Technology

Infusion systems for infusing one or more fluids into a patient are commonplace in modern healthcare environments. Infusion systems can include an infusion pump and an infusion set. A pump cassette can be loaded into certain infusion pumps. The infusion set can include the pump cassette and other components that facilitate fluid to flow from a container to a patient.

Using infusion sets can perform better and/or be safer to use under certain conditions. Improved methods and components for providing information associated with an infusion set to an infusion pump are desired.

In certain healthcare environments, multiple infusion lines can be connected to a single patient. Improved infusion line identification would be desirable.

### SUMMARY

The innovations described in the claims each have several aspects, no single one of which is solely responsible for its desirable attributes. Without limiting the scope of the claims, some prominent features of this disclosure will now be briefly described.

Various pump cassettes with a radio frequency identification tag and related infusion pumps, infusion systems, and methods are described herein. Although many of the examples are described in the context of an infusion pump, the technology described herein can be applied to any other suitable medical devices and/or non-medical devices in addition to infusion pumps, and in other environments. Other medical devices (instead of or including an infusion pump), or non-medical devices, or any combination thereof can be implemented in place of infusion pumps disclosed herein as suitable.

In various embodiments, a pump cassette includes a radio frequency identification tag. The radio frequency identification tag can store a variety of useful infusion, such as usage information associated with the pump cassette and/or a unique identifier of the pump cassette. In certain embodiments, the radio frequency identification tag can be embedded in a component of the pump cassette. For example, the radio frequency identification tag can be embedded in a flow stop valve of the pump cassette. An infusion pump can include a radio frequency identification reader having an antenna aligned with an antenna of the radio frequency tag when the pump cassette is loaded into the infusion pump. The radio frequency identification tag of the pump cassette can provide useful information to perform safety checks or otherwise provide information to control infusion.

One aspect of this disclosure is a pump cassette for fluid delivery. The pump cassette includes a housing dimensioned to engage with an infusion pump; a fluid path within the housing, the fluid path configured to receive fluid from a fluid source and to deliver the fluid to tubing of an infusion line; and a radio frequency identification tag embedded in a component of the pump cassette.

The radio frequency identification tag can be surrounded by molding material of the component. The component can be a mechanical component. The component can be a flow stop valve.

The radio frequency identification tag can store an identifier uniquely identifying the pump cassette. The radio frequency identification tag can store pairing information for wireless communication with a light set. The radio frequency identification tag can store cassette usage information. The radio frequency identification tag can store information identifying one or more pump cassette parameters.

The fluid path can include an air trap. The fluid path can include a pumping chamber. The fluid path can be U shaped. The pump cassette can independently control pumping for a primary infusion and a secondary infusion. The pump cassette can deliver a set volume of the fluid with each pumping cycle.

Another aspect of this disclosure is an infusion system for fluid delivery. The infusion system comprising includes an infusion pump, fluid tubing, and a pump cassette loaded into the infusion pump. The pump cassette is configured to deliver fluid to the fluid tubing. The pump cassette includes a component with an embedded radio frequency identification tag.

The infusion pump can include a radio frequency identification reader aligned with the radio frequency identification tag when the pump cassette is loaded into the infusion pump. An antenna of the radio frequency identification tag can be aligned with an antenna of the infusion pump. The pump cassette can include any suitable combination of features of the pump cassettes disclosed herein.

Another aspect of this disclosure is an infusion pump for fluid delivery. The infusion pump includes a housing configured to receive a pump cassette, and an antenna configured to communicate with a radio frequency identification tag of the pump cassette when the pump cassette is engaged with the housing. The antenna is arranged to be aligned with a flow stop valve of pump cassette when the pump cassette is engaged with the housing. The infusion pump can include a processor configured to perform a safety check based on information from the radio frequency tag read by way of the antenna, and to cause infusion of fluid to a patient based on passing the safety check.

Another aspect of this disclosure is a method of manufacturing a pump cassette. The method includes positioning a radio frequency identification assembly on a structure of a component of a pump cassette, covering the radio frequency identification assembly with a molding material, and connecting the component with another component of the pump cassette.

The component can be a flow stop valve. The method can further include storing an identifier uniquely identifying the pump cassette to memory of the radio frequency identification assembly. Covering the radio frequency tag with the molding material can include positioning a cap portion over the radio frequency identification assembly to engage with a cavity.

Another aspect of this disclosure is a method of operating an infusion pump. The method includes reading information from a radio frequency identification tag embedded in a component of a pump cassette; and controlling infusion, by the infusion pump, based on the information from the radio frequency tag.

The method can include writing, by the infusion pump, to the radio frequency identification tag during infusion. The method can include writing information indicative of a start time of infusion to the radio frequency identification tag. The method can include writing information indicative of a medication delivered to a patient to the radio frequency identification tag.

Controlling infusion can include verifying a pump cassette based on the information. Controlling infusion can include performing a safety check related to previous usage of the pump cassette. Controlling infusion can include performing a safety check related to a condition of the pump cassette.

Another aspect of this disclosure is a pump cassette for fluid delivery. The pump cassette includes a housing dimensioned to engage with an infusion pump; a fluid path within the housing, the fluid path configured to receive fluid from a fluid source and to deliver the fluid to tubing of an infusion line; and a radio frequency identification tag storing usage information associated with the pump cassette.

The usage information can identify an active medication delivered using the pump cassette. The usage information can identify a time at which the pump cassette started delivering the fluid. The usage information can identify a previous infusion pump in which the pump cassette was used.

The radio frequency identification tag can store an identifier uniquely identifying the pump cassette. The radio frequency identification tag can store pairing information for wirelessly communicating with a light set.

Another aspect of this disclosure is an infusion system for fluid delivery. The infusion system includes an infusion pump, fluid tubing, and a pump cassette loaded into the infusion pump. The pump cassette is configured to deliver fluid to the fluid tubing. The pump cassette includes a radio frequency identification tag storing usage information associated with the pump cassette.

Another aspect of this disclosure is a method of operating an infusion pump. The method includes reading usage information from a radio frequency identification tag of a pump cassette, performing a safety check using the usage information, and delivering a fluid to an infusion line in response to passing the safety check.

The radio frequency identification tag can be embedded in a component of the pump cassette. The radio frequency identification tag can be embedded in a flow stop valve of the pump cassette. The radio frequency identification tag can be integrated with a flow stop valve of the pump cassette.

The method can include writing to the radio frequency identification tag during infusion. The method can include writing information indicative of an infusion start time writing to the radio frequency identification tag. The method can include writing information indicative of an active drug in the fluid to the radio frequency identification tag.

The safety check can relate to prior usage of the pump cassette. The safety check can relate to an expiration date of the pump cassette. The safety check can relate to whether one or more particular drugs have been infused using the pump cassette. The safety check can relate to an infusion start time associated with the pump cassette.

Various light sets and related tubing sets, infusion sets, and methods are described herein. Although many of the examples are described in the context of an infusion set, the technology of the lights sets described herein can be applied to any other suitable tubing set, such as another type of medical tubing set or a non-medical tubing set, and in other environments.

In various embodiments, a tubing set includes fluid tubing and a light set attached to the fluid tubing. The light set can include a light source, an antenna, a wireless communication chip in communication with the antenna, a battery, and a battery contact. An insulator tape flag can be positioned between the battery and a battery contact. The light set can be activated by moving the insulator tape flag from between the battery and the battery contact. For example, straightening the tube can cause the insulator tape flag to be moved such that the light set is activated. In some instances, the tubing set can be an infusion set that includes a pump cassette with a radio frequency identification tag. The radio frequency identification tag can store pairing information for the light set, such as an address of the light set. The tubing sets disclosed herein can enable improved line identification and/or status indicators. Tubing sets disclosed herein can be inactive before use and be activated as a tubing set is arranged for use without additional user action.

An aspect of this disclosure is a tubing set that includes fluid tubing; a light set attached to the fluid tubing, the light set comprising a light source, an antenna, a wireless communication chip in communication with the antenna, a battery, and a battery contact; and an insulator tape flag positioned between the battery and the battery contact.

The tape and the light set can be attached to the fluid tubing such that straightening the fluid tubing causes the insulator tape flag to move so that the battery is electrically connected to the battery contact. The light set can include tape for a clinician to mark.

The wireless communication chip can be a Bluetooth Low Energy chip. The wireless communication chip can be configured to provide a wireless personal area network signal to the antenna.

The light source can include a light emitting diode. The light source can include at least two light emitting diodes configured to provide light of different colors. The light source is configured to provide light of at least two different colors.

The tubing set can be a medical tubing set. The tubing set can be an infusion set.

Another aspect of this disclosure is a method of activating a light set attached to fluid tubing. The method includes straightening the fluid tubing such that an insulator tape flag is moved from between a battery of the light set and a battery contact of the light set to thereby activate the light set. The light set can include a wireless communication component.

Another aspect of this disclosure is an infusion set that includes fluid tubing; a light set attached to the fluid tubing, the light set includes a light source and a wireless communication component; and a pump cassette includes a radio frequency identification tag storing pairing information for wireless communication with the wireless communication component of the light set.

The infusion set can include a second light set attached to the fluid tubing. The radio frequency identification tag can be embedded in a component of the pump cassette, such as a flow stop valve. An insulator tape flag can be positioned between a battery of the light set and a battery contact of the light set. The pairing information can include a media access control address of the light set.

Another aspect of this disclosure is a method of establishing wireless communication with a light set in an infusion environment. The method includes reading, by an infusion pump, pairing information from a radio frequency identification tag of a pump cassette loaded into the infusion pump; and establishing, by the infusion pump, a wireless link with the light set using the pairing information.

Another aspect of this disclosure is a method of illuminating medical tubing. The method includes reading pairing information for a light set from a radio frequency identification tag of a pump cassette loaded into an infusion pump, wherein the light set is attached to medical tubing, and wherein the medical tubing is in fluid communication with the pump cassette; establishing a wireless connection with the light set using the pairing information; and wirelessly transmitting information to the light set to cause a light source of the light set to illuminate.

The pairing information can include an authentication key. The infusion pump can perform said reading and said wirelessly transmitting. The light source can include a light emitting diode. The light set can include a Bluetooth Low Energy chip.

Another aspect of this disclosure is a method of illuminating an infusion set to provide an infusion status indicator. The method includes determining, by an infusion pump, a status associated with an infusion; wirelessly transmitting, by the infusion pump, a first instruction to a plurality of light sets of the infusion set that are connected to a fluid pathway of the infusion set, wherein the first instructions cause a respective light source of each of the light sets to illuminate; and wirelessly transmitting, by the infusion pump, a second instruction to at least one light set of the plurality of light sets, wherein the second instruction causes the light source of the one light set to illuminate differently than the first instructions to indicate a different status associated with infusion.

The second instruction can cause the light source of the one light set to provide a different color of light than the first instruction. The second instruction can cause the light source of the one light set to provide light for a different duration of time than the first instruction. The second instruction can cause the light source of the one light set to provide flashes of light at a different interval than the first instruction. The second instruction can be associated with an issue with infusion.

There can be a direct wireless link between the infusion pump and each of the light sets. The infusion pump can independently control each of the light sets.

These and other embodiments are described in greater detail below with reference to the Figures. For purposes of summarizing the disclosure, certain aspects, advantages and novel features of the innovations have been described herein. It is to be understood that not necessarily all such advantages may be achieved in accordance with any particular embodiment. Thus, the innovations may be embodied or carried out in a manner that achieves or optimizes one advantage or group of advantages as taught herein without necessarily achieving other advantages as may be taught or suggested herein.

### BRIEF DESCRIPTION OF THE DRAWINGS

Embodiments of this disclosure will be described, by way of non-limiting example, with reference to the accompanying drawings.

FIG. 1 is a schematic block diagram of an infusion environment in which pump cassettes and/or infusion sets and/or light sets according to embodiments can be deployed.

FIG. 2 illustrates an example infusion pump.

FIGS. 3A and 3B illustrate a pump cassette with a radio frequency identification (RFID) tag integrated with a flow stop valve according to an embodiment.

FIG. 3C illustrates part of an infusion pump with an RFID reader.

FIGS. 4A to 4D illustrate a flow stop valve of a pump cassette with an embedded RFID tag according to an embodiment at various stages of manufacture.

FIG. 5 illustrates a pump cassette with an RFID tag attached to a curved portion of a pump cassette body outside of a pump chamber according to an embodiment.

FIG. 6 illustrates a pump cassette with an RFID tag attached to a pump cassette body facing a splash shield according to an embodiment.

FIG. 7 illustrates a pump cassette with an RFID tag attached to an outside wall of an air trap of a pump cassette according to an embodiment.

FIG. 8 illustrates an example pump cassette.

FIGS. 9A and 9B illustrate another example pump cassette.

FIG. 10 illustrates the pump cassette of FIGS. 9A and 9B loaded into an example infusion pump.

FIG. 11 illustrates another example pump cassette.

FIG. 12A illustrates an example infusion set according to an embodiment.

FIG. 12B illustrates another example infusion set according to an embodiment.

FIG. 12C illustrates another example infusion set according to an embodiment.

FIG. 13 illustrates an example infusion environment with multiple infusion lines.

FIG. 14 is a schematic block diagram of an infusion set with light sets arranged to wirelessly communicate with an infusion pump according to an embodiment.

FIG. 15A illustrates an assembled light set according to an embodiment.

FIG. 15B illustrates an exploded view of the light set of FIG. 15A.

FIG. 16A illustrates a light set according to an embodiment before activation. FIG. 16B illustrates the light set of FIG. 16A after activation.

FIGS. 17A to 17E illustrate a light set according to an embodiment.

FIGS. 18A and 18B illustrate another light set according to an embodiment.

### DETAILED DESCRIPTION OF CERTAIN EMBODIMENTS

The following detailed description of certain embodiments presents various descriptions of specific embodiments. However, the innovations described herein can be embodied in a multitude of different ways, for example, as defined and covered by the claims. In this description, reference is made to the drawings where like reference numerals and/or symbols can indicate identical or functionally similar elements. It will be understood that elements illustrated in the figures are not necessarily drawn to scale. Moreover, it will be understood that certain embodiments can include more elements than illustrated in a drawing and/or a subset of the elements illustrated in a drawing. Further, some embodiments can incorporate any suitable combination of features from two or more drawings. The headings provided herein are for convenience only and are not intended to affect the meaning or scope of the claims.

It can be desirable to provide information associated with infusion sets to infusion pumps. Infusion pumps can perform safety checks, control infusion, and/or implement other functionality with such information. This disclosure provides technical solutions related to providing information stored in a pump cassette to an infusion pump and/or to storing information associated with an infusion set to a component of the pump cassette.

Aspects of this disclosure relate to a pump cassette with a radio frequency identification (RFID) tag. The RFID tag can store a variety of useful information. For example, the RFID tag can store an identifier uniquely identifying the pump cassette, pump cassette usage information, information identifying one or more pump cassette parameters, pairing information for wireless communication with a light set of an infusion set, the like, or any suitable combination thereof. Such information can be read by an RFID reader of an infusion pump. The RFID reader can be aligned with the RFID tag when the pump cassette is loaded into the infusion pump. The infusion pump can read from the RFID tag and/or write to the RFID tag before infusion, during infusion, and/or after infusion. With information from the RFID tag, the infusion pump can perform one or more safety checks and/or otherwise use information from the RFID tag. In some applications, a pump cassette can include two or more RFID tags. Related infusion sets, infusion pumps, infusion systems, and methods are provided.

In certain embodiments, the RFID tag can be embedded into a component of the pump cassette. The component can be a flow stop valve, for example. With an RFID tag embedded into a component of the pump cassette, the pump cassette can store information and wirelessly communicate with one or more other devices with the same form factor and mechanical functionality as pump cassettes without an embedded RFID tag.

Although embodiments may be discussed with reference to RFID tags, any suitable principles and advantages disclosed herein with reference to an RFID tag can be implemented in association with any other suitable wireless communication component comprising an antenna and a wireless communication chip.

As discussed above, line identification is useful in infusion environments with multiple infusion lines. Reducing and/or eliminating time, effort and/or errors associated with manual line identification is generally desirable. A technical solution involving simple setup of a light set for line identification without additional actions by a caregiver would be desirable.

Aspects of this disclosure relate to light sets for infusion sets. The light sets can be provided in an inactive state. For example, an electrically insulating tape flag can be positioned between a battery and a battery contact of a light set so that the light set is inactive. The infusion set can be packaged with the light set in the inactive state. A light set can be activated by straightening tubing of the infusion set or another operation that would be performed by a caregiver to setup the infusion set for infusion. As an example, straightening tubing can move the insulating tape flag from between the battery and the battery contact to thereby bring the battery in contact with the battery contact to activate the light set. The light set can include other features such as one or more of protection of electronics for desired use, an attachment method for various diameters of tubing and/or connectors, ease of assembly, visibility of illumination from all directions, being sterilizable with processes for sterilizing medical devices, computability with MRI, a sufficient battery life sufficient for desired uses, and a process for pairing for wireless communication with an infusion pump.

A light set can be illuminated based on an instruction or command from an infusion pump. The infusion pump can wirelessly communicate with the light set. The infusion pump can illuminate the light set for line identification, a status identifier, an alarm, the like, or any suitable combination thereof.

An infusion set can include a pump cassette with an RFID tag and one or more light sets. The RFID tag of the pump cassette can store pairing information, such as a media access control address (MAC address), for wirelessly communicating with a light set. An infusion pump can read the pairing information from the RFID tag of the pump cassette and establish a wireless link with the light set using the pairing information.

### Infusion Environment

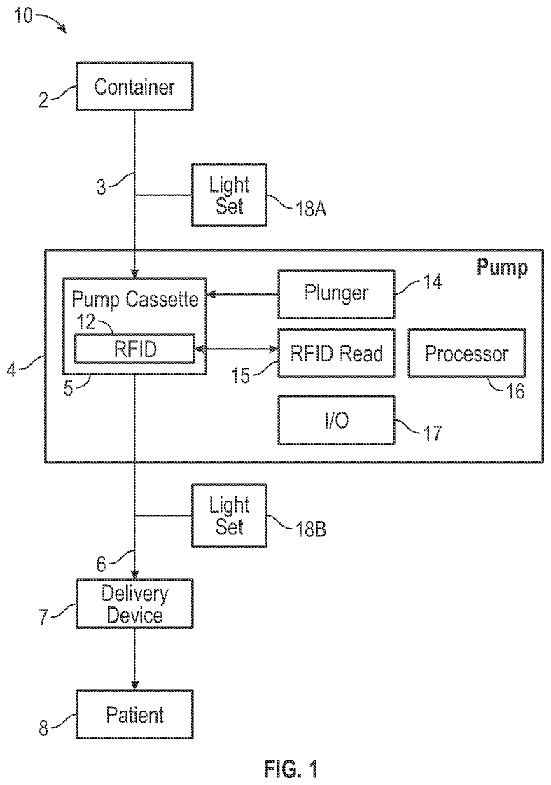

FIG. 1 illustrates a schematic block diagram of an example infusion environment 10 in which pump cassettes, infusion sets, and/or light sets disclosed herein can be deployed. In the infusion environment 10, an infusion system comprises an infusion container 2 storing a medical fluid, a primary line 3, an infusion pump 4, a pump cassette 5 loaded into the infusion pump 4, an infusion line 6, and a delivery device 7. The infusion system may comprise an infusion system such as the Plum™ infusion system or any other suitable type of infusion system. The infusion system provides an infusion fluid to a patient 8.

The infusion container 2 comprises a container for delivering an infusion fluid, such as intravenous (IV) fluid or a drug, to the patient 8. The infusion container 2 is an example of a fluid source. One or more fluid tubes can be connected between the infusion container 2 and the delivery device 7. Infusion fluid flows through fluid tubing from the infusion container 2, through the pump cassette 5, and through the delivery device 7 to the patient 8.

The pump cassette 5 has a housing dimensioned to engage with the infusion pump 4. Within the housing, the pump cassette 5 has a fluid pathway. The pump cassette 5 includes an RFID tag 12. The pump cassette 5 can include any suitable combination of features of the pump cassettes disclosed herein. The RFID 12 can be implemented in accordance with any suitable principles and advantages of the RFIDs of pump cassettes disclosed herein.

The illustrated pump 4 includes a plunger 14, an RFID reader 15, a processor 16, and an input/output device 17. The plunger 14 can facilitate pumping of infusion fluid from the infusion container 2 through the pump cassette 5. The RFID reader 15 can read data from the RFID tag 12 of the pump cassette 5 when the pump cassette 5 is loaded into the infusion pump 4. The RFID reader 15 can write information to the RFID tag 12 when the pump cassette is loaded into the infusion pump 4. An antenna of the RFID reader 15 can be aligned with an antenna of the RFID tag 12 when the pump cassette 5 is loaded into the infusion pump 4. The processor 16 can be programmed to control infusion. The processor 16 is configured to process data read from the RFID tag 12. The processor 16 is configured to cause data to be written to the RFID tag 12. The processor 16 can perform one or more safety checks, control infusion, alarm, the like, or any suitable combination thereof based on information read from the RFID tag 12.

The illustrated infusion pump 4 includes an input/output device 17 that can receive information from a clinician and/or provide information to a clinician. For example, the input/output device 17 allows a clinician to input information such as: medication information regarding the infusion fluid being delivered from the infusion container 2; infusion information regarding the infusion of the infusion fluid being delivered from the infusion container 2; the selection of settings for the processor 16 to apply in using programming code containing the algorithm(s); or other information that is pertinent to the infusion. The input/output device 17 may allow a clinician to select and/or confirm a user-inputted medication infusion program to be applied by the infusion pump 4. The input/output device 17 may further output information to the clinician. In some other embodiments, any of the information inputted into the input/output device 17 may be pre-installed into the programming code and/or the processor 16. In another embodiment, the information may be remotely programmed into processor 16 and/or memory from a remote computer or the input/output device 17 may be a remote and/or portable computer.

The delivery device 7 can comprise a patient vascular access point device for delivering infusion fluid from the infusion container 2 to the patient 7. The delivery device 7 can comprise a needle, a catheter, a cannula, or any other suitable type of delivery device. In certain applications, the delivery device 7 can also be used for extracting blood from the patient 8.

Light sets 18A and 18B can be attached and/or integrated with fluid tubing and/or other components of the infusion system. As illustrated, a first light set 18A is attached to the primary line 3 and a second light set 18B is attached to an infusion line 6. The light sets 18A and 18B can illuminate for line identification, to provide a status indicator, to alarm, the like, or any suitable combination thereof.

Infusion pumps disclosed herein can be bedside pumps, ambulatory pumps, or any other suitable infusion pumps. Infusion pumps disclosed herein can be stationary pumps or portable pumps. Although embodiments may be discussed with reference to infusion pumps, pump cassettes in accordance with any suitable principles and advantages disclosed herein can be implemented with any suitable pump configured to receive a pump cassette.

Although embodiments may be discussed with reference to infusion sets, light sets in accordance with any suitable principles and advantages disclosed herein can be implemented in any suitable tubing set. Such tubing sets include a variety of medical tubing sets.

### Infusion Pump

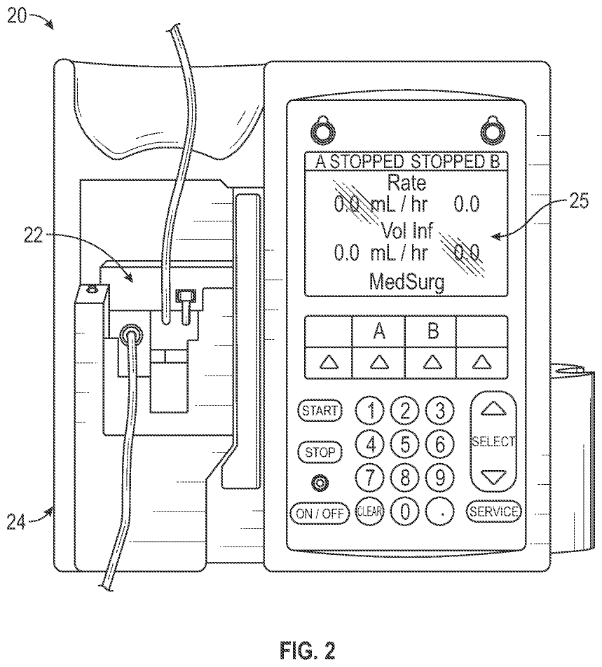

FIG. 2 illustrates an example infusion pump 20. The infusion pump 20 comprises an opening 22 configured to receive a pump cassette. A door 24 of the infusion pump 20 can be opened for loading the pump cassette as illustrated in FIG. 2. The door 24 can be closed to secure the pump cassette. The infusion pump 20 may include a display 25 configured to allow the user to input parameters for an infusion or to display error messages, error codes, suggested actions, the like, or any suitable combination thereof. Within a housing, the infusion pump 20 can include a memory, at least one processor, a plunger, and other components. The memory can store computer-executable instructions the at least one processor can execute to cause the infusion pump 20 to control infusion of an infusion fluid. The infusion pump 20 can include an RFID reader configured to read an RFID tag of a pump cassette loaded into the infusion pump 20. Although FIG. 2 illustrates a single channel infusion pump, any suitable principles and advantages disclosed herein can be applied to a multi-channel pump.

Pump Cassettes with RFID and Related Infusion Systems

As discussed above, a pump cassette can include an RFID tag. An RFID tag includes an antenna and an integrated circuit. The antenna can wirelessly transmit and/or receive radio frequency signals. The integrated circuit can store information and facilitate wireless communication. The antenna and the integrated circuit can be positioned on a common substrate. The antenna can be a coil antenna. Some RFID tags are passive tags that reflect back energy from an RFID reader. Passive RFID tags typically do not include a battery. Passive RFID tags can wirelessly communicate at a low frequency (LF) in a range from 124 kHz to 134 kHz, at a high frequency (HF) of 13.56 MHz, or an ultra high frequency (UHF) in a range from 865 MHz to 960 MHz, for example. Certain passive RFID tags can be embedded in components. Active RFID tags can include a battery to power the RFID tag. An active RFID tag for a pump cassette can have a battery life sufficient to power the RFID tag for the useful life of the pump cassette. An active RFID tag can wirelessly communicate signals with frequencies of 433 MHz, 915 MHz, or 2.45 GHz, for example. Semi-passive RFID tags can include a battery and transmit with the energy provided by an RFID reader. Any suitable RFID tag can be integrated in a pump cassette for a particular application.

Storing information to an RFID tag of the pump cassette can reduce a risk of low connectivity and/or lack of connectively with a network for an infusion pump. The RFID tag can store a variety of information that can be used to determine one or more infusion parameters and/or to perform one or more safety checks. Example information stored in an RFID tag of a pump cassette will now be discussed. Any of this information can be stored in any of the RFID tags of pump cassettes disclosed herein as suitable.

The RFID tag of the pump cassette can store set control information. The set control information relates to information about an infusion set. The set control information can include one or more of: a list identifier, manufacturing information (e.g., a lot number, a manufacturing date, batch information, etc.), an expiration information (e.g., an expiration date or a number of months until expiration), a purpose for the set (e.g., standard IV, epidural, blood, light sensitive oncology, sub-cutaneous, etc.), a priming volume (e.g., in uL), or the like. An infusion pump can perform a safety check based on the set control information. As one example, the set control information can be read by an infusion pump and used to verify that the infusion set is not expired before using the infusion set. Any suitable set control information can be used to perform a variety of other safety checks.

The RFID tag of the pump cassette can store usage information associated with the pump cassette. The usage information can identify an active drug infused with the infusion set, an infusion start time, an amount of time used, a time use limit, a pump cassette last used time, or the like. The active drug information can be read by an infusion pump and the infusion pump can determine whether the active drug is a high alert medication (e.g., dopamine) where there are limitations on infusion set usage after infusing the high alert medication. For example, the infusion pump could disable infusion of a different drug with an infusion set that was previously used with a high alert medication. The infusion pump can make such a determination from information read from the RFID tag of the pump cassette without being connected to a network. The infusion start time information can be read by the infusion pump and the infusion pump can alarm in response to determining that a threshold amount of time has passed since the start of infusion. The pump cassette last used time information can be read by the infusion pump and the infusion pump can alarm in response to determining that a threshold amount of time has passed since the last used time. The usage information can provide information to the infusion pump in case the pump cassette was previously used in another pump and moved to the infusion pump.

The RFID tag of the pump cassette can store information about set features. The set features can provide information about a variety of features of an infusion set. Set feature information can include proximal feature information, pump cassette information, distal feature information, or any suitable combination thereof. The proximal feature information can identify whether the infusion set includes and/or additional information regarding (e.g., a capacity, number of, and/or a type of) one or more of the following: drip chamber, burette, filter, clamp, clave, tubing bore, tubing type, or the like. The pump cassette information can store one or more of the following: cassette type information, air trap information, or secondary port information. The cassette type information can identify the type of the cassette, such as a standard cassette, an ambulatory cassette, a pediatric cassette, a newborn intensive care unit (NICU) cassette, or the like. The air trap information can identify whether there is an air trap and/or the size of the air trap. The secondary port information can identify whether there is a secondary port and/or a type of the secondary port, such as a closed connector (e.g. a Clave™ connector or a Microclave™ connector), a pre-pierced septum, or the like. The distal feature information can identify whether the infusion set includes and/or additional information regarding (e.g., a capacity, number of, and/or a type of) one or more of the following: clamp, filter, clave, tubing bore, tubing type, connector, priming cap, or the like. Any suitable set feature information can be used to verify and/or adjust one or more infusion parameters.

The RFID tag of the pump cassette can store a unique identifier of the pump cassette and/or the infusion set. For instance, the unique identifier can be a digital signature. The unique identifier can be written to the RFID tag of the pump cassette during manufacturing. The unique identifier can be written to the RFID tag of the pump cassette before the pump cassette is packaged with other components of an infusion set. The infusion pump can determine whether the pump cassette is an authentic pump cassette based on the unique identifier. This can help prevent counterfeit pump cassettes from being used.

The RFID tag of the pump cassette can store pairing information for another device of an infusion system. For example, the RFID tag can store pairing information for a light set. The pairing information can include a media access control address (MAC address) for wirelessly communicating with light set.

An example infusion workflow will now be described. A pump cassette can be loaded into an infusion pump. A door of the infusion pump can be closed. An RFID reader of the infusion pump can read information from the RFID tag of the pump cassette.

The infusion pump can perform one or more safety checks using the information read from the RFID tag of the pump cassette. The infusion pump can authenticate an infusion set and/or pump cassette by checking whether the RFID tag stores a valid digital signature.

The infusion pump can determine whether the pump cassette has been previously used. The infusion pump can determine whether a high risk drug was infused using the pump cassette. If a high risk drug was previously infused using the pump cassette and the infusion pump is programmed to infuse the same drug, the infusion pump can determine to proceed with infusion. If a high risk drug was previously infused using the pump cassette and the infusion pump is programmed to infuse a different drug, the infusion pump can determine to not proceed with infusion. The infusion pump can determine whether the pump is cassette still valid to run (e.g., if it has been less than a pre-determined time period, for example 72 hours, since the pump cassette was first used for infusion). If so, the infusion pump can determine to proceed with infusion.

The infusion pump can check pump parameters. The infusion pump can determine whether the pump cassette has expired based on information read from the RFID tag of the pump cassette. If the infusion pump determines that the pump cassette is expired, the infusion pump can decide not to proceed with infusion.

A user can program the infusion pump. The infusion pump can then check safety and/or infusion parameters based on information read from the RFID tag of the pump cassette to determine whether to proceed with infusion. For example, the infusion pump can check if a filter is present and/or a filter size. Infusions for certain medications that should not be used with a filter can be prevented when the RFID tag indicates the infusion set includes a filter. Infusions for certain medications that should be used with a filter can be prevented when the RFID tag indicates the infusion set does not include a filter. As another example, the infusion pump can adjust pressure settings based on tubing bore size. As one more example, the infusion pump can check for a shallow mechanism stroke.

The RFID reader of the infusion pump can write information identifying a start time of an infusion and/or an active medication for infusion to the RFID tag of the pump cassette while the pump cassette is loaded into the infusion pump. Then infusion can begin.

While the infusion pump is running, the infusion pump can write to the RFID tag of the pump cassette. A user can add a secondary medication for infusion. The infusion pump can write information identifying the secondary medication to the RFID tag of the pump cassette. The infusion pump can store information indicative of when the secondary infusion ends and the primary infusion flushes to the RFID tag. The active medication information on the RFID tag can be updated.

The infusion pump can determine that the pump cassette expires during infusion. The infusion pump can alarm and continue to pump in response to determining pump cassette expiration.

The infusion pump can determine that the cassette times out. Cassette time outs can include hard limits, such as the pump cassette running for more than 72 hours or more than 4 hours if blood is being infused. Cassette time outs can include soft limits, such as drug based limits (e.g., propofol is in any infusion set for more than 12 to 24 hours can be set in a drug library). The infusion pump can alarm and continue to pump in response to determining the pump cassette times out.

Example pump cassettes with RFID tags are disclosed. The RFID tag can be positioned such that an antenna of the RFID tag and an antenna of an infusion pump are oriented to provide efficient energy transfer. For example, the antenna of the RFID tag of the pump cassette can be aligned with an antenna of an RFID reader of an infusion pump when the pump cassette is loaded into an infusion pump. It can be desirable to implement the RFID tag with relatively few changes to the pump cassette. It can also be desirable to implement the RFID reader with relatively few changes to an infusion pump. As one example, an RFID tag integrated with a flow stop valve of a pump cassette can be implemented with relatively few changes to both the pump cassette and the infusion pump.

The RFID tags discussed herein can have sufficient memory (e.g., at least 512 bytes of storage or at least 1024 bytes of storage) to store any suitable information discussed herein. The RFID tags discussed herein can retain data through a sterilization process (e.g., 50 kGy gamma ray sterilization). The RFID tags can have a small form factor.

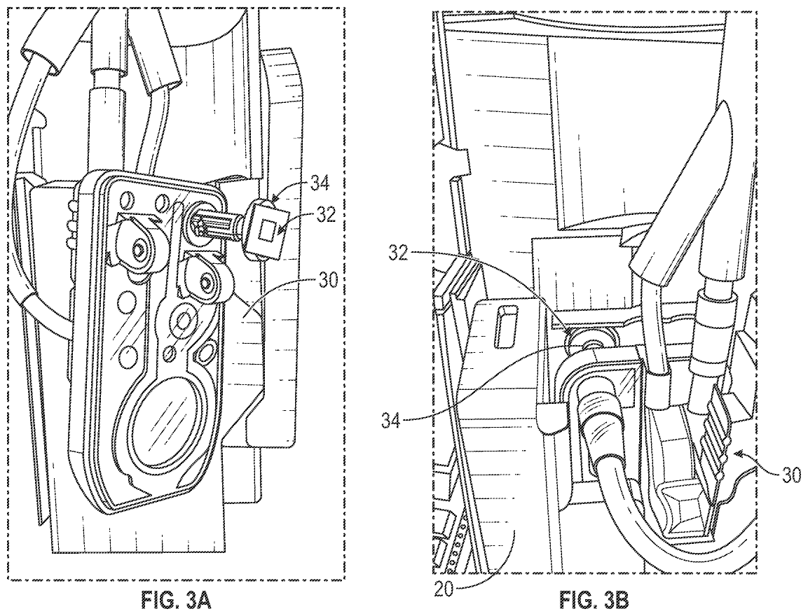

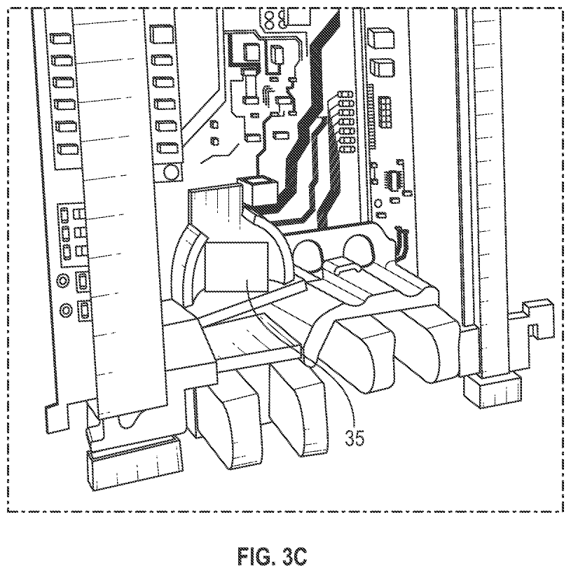

FIGS. 3A and 3B illustrate a pump cassette 30 with an RFID tag 32 integrated with a flow stop valve 34. FIG. 3C illustrates part of an infusion pump with an RFID reader 35.

As shown in FIG. 3A, the pump cassette 30 includes the RFID tag 32 attached to the flow stop valve 34. With the RFID tag 32 integrated with the flow stop valve 34, there can be sufficient space to allow the flow stop valve 34 to pass a splash shield. An antenna of an RFID reader of an infusion pump can be positioned behind a regulator closer and shaft and be aligned with an antenna of the RFID tag 32 for wirelessly communicating with the RFID tag 32. Such alignment can result in efficient and/or maximum energy transfer between the antennas of the RFID tag 32 and the antenna of the RFID reader.

FIG. 3B shows on opposite side of the pump cassette 30 relative to FIG. 3A. In FIG. 3B, the pump cassette 30 is loaded into the infusion pump 20. Parts of the infusion pump 20 are not shown in FIG. 3B to better illustrate the pump cassette 30.

An RFID reader can be included in an infusion pump. Such an RFID reader can wirelessly communicate with the RFID tag of the pump cassette without changing existing workflows. An RFID reader of an infusion pump can write to the RFID tag of the pump cassette as an infusion progresses. Accordingly, the infusion pump can store usage information associated with the pump cassette to the RFID tag of the pump cassette during infusion. The RFID reader of the infusion pump can write to the RFID tag of the pump cassette before infusion and/or after infusion.

A start time of the initial infusion can be stored in the RFID tag of the pump cassette by the RFID reader of the infusion pump. This can enable additional safety checks, such as blood sets only being used within a certain amount of time (e.g., 4 hours) from the start of infusion. As another example, a safety check with related to sets with variability in duration (e.g., based on a particular medication) can be performed. An RFID reader in an infusion pump can be used to check whether an infusion set has been used with a medication that could present an issue with an infusion with the infusion pump. For example, if the infusion set has been used with other pumps, a set change can be prompted to prevent unwanted medication (e.g., heparin, dopamine, etc.) from being flushed into a patient. Storing information about an active drug infused by an infusion set in an RFID tag of the pump cassette enables cross-checking by an infusion pump even when the infusion pump is disconnected from a network.

FIG. 3C shows a portion of the infusion pump 20 that is within a housing of the infusion pump 20. The infusion pump 20 includes an antenna 35 that is positioned for reliable wireless communication with the RFID tag 32 of a pump cassette 30 loaded into the infusion pump. The antenna 35 can be aligned with an antenna of the RFID tag 32 that is integrated with the flow stop valve 34. The antenna 35 can be aligned with an antenna of an RFID tag embedded within a flow stop valve. An RFID reader can include the antenna 35. The antenna 35 can be used to read from the RFID tag 32 when the pump cassette 30 is loaded into the infusion pump 20. The antenna 35 can be used to write to the RFID tag 32 when the pump cassette 30 is loaded into the infusion pump 20. The infusion pump 20 can read from and/or write to the RFID tag 32 during an infusion where fluid is provided to a patient from the pump cassette 30.

An RFID tag can be embedded in a component of the pump cassette. The RFID tag can be embedded in a mechanical component of the pump cassette. Molding material of the component can surround the RFID tag. The component can be sized such that the RFID tag can be embedded therein. The RFID tag can be a passive, embeddable RFID tag. The component can be located such that when the pump cassette is loaded into the infusion pump, the infusion pump has space for an antenna aligned with an antenna of the RFID tag.

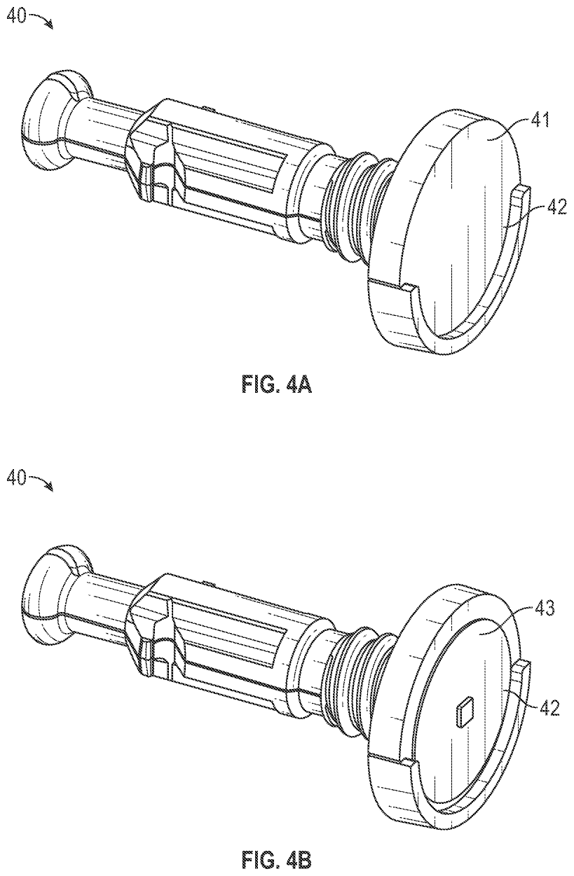

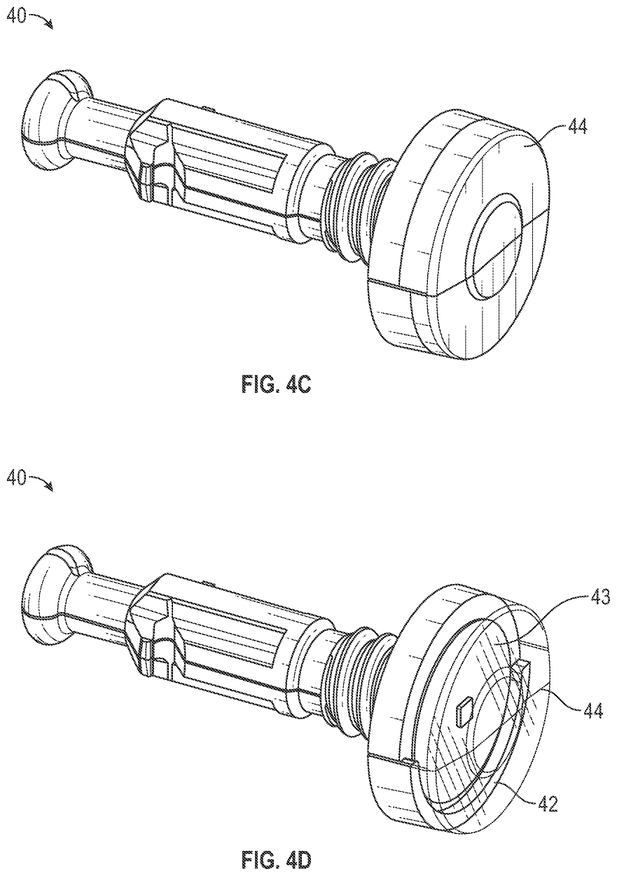

FIGS. 4A to 4C illustrate a flow stop valve with an embedded RFID tag as various stages of manufacture. FIG. 4D illustrates the flow stop value of FIG. 4C with transparent cap so that a position of the RFID tag embedded in the flow stop valve can be seen.

By embedding the RFID tag into the flow stop valve, the pump cassette can have the same mechanical function and the same footprint. This can result in few changes to the pump cassette and/or speed up regulatory approval. In addition, no extra physical space of the pump cassette is consumed by embedding the RFID tag in the flow stop valve. An infusion pump can also have space within a housing for an antenna aligned with an antenna embedded within the flow stop valve such that an RFID reader with efficient energy transfer can be implemented with relatively few modifications of the infusion pump.

FIG. 4A illustrates a body portion 40 of a flow stop valve. The body portion 40 can be an injection molded part. The body portion 40 can be formed by a first injection. The body portion 40 can be formed of polyethylene terephthalate glycol (PETG) or modified PETG. The body portion 40 can be formed of any other suitable medical grade molding material. An RFID assembly can be placed onto a surface 41 of the body portion 40 while the body portion 40 is positioned in an ejection side of a tool. A cavity 42 of the body portion is also shown in FIG. 4A.

FIG. 4B illustrates an RFID assembly 43 placed onto the surface 41 of the body portion 40. The RFID assembly 43 can comprise an antenna and an integrated circuit. The RFID assembly 43 can be sized to fit on the surface of the body portion 40. The RFID assembly 43 can have a diameter in a range from about 6 millimeters (mm) to 10 mm, for example. The diameter of the RFID assembly 43 can be approximately 8 mm in certain applications. The RFID assembly 43 can be adhesive backed. Accordingly, the RFID assembly 43 can be attached to the surface 41 of the body portion in FIG. 4B.

FIG. 4C illustrates a cap portion 44 over the RFID assembly. The cap portion 44 can engage with a cavity 42 of the body portion 40. The cap portion 44 can be an overmold injection. As shown in FIG. 4C, the RFID assembly 43 is embedded in the flow stop value. The RFID assembly 43 is surrounded by molding material of the flow stop valve. In FIG. 4D, the cap portion 44 is shown as being clear to show the location of the RFID assembly 43 in the flow stop valve.

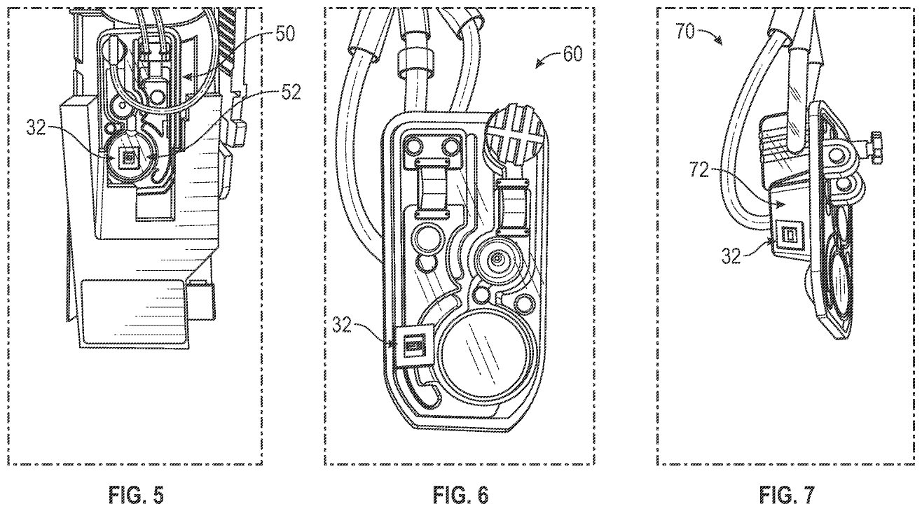

In some other applications, an RFID tag can be integrated with a pump cassette in any suitable location. FIGS. 5 to 7 illustrate an RFID tag integrated with the pump cassette at various locations.

FIG. 5 illustrates a pump cassette 50 with an RFID tag 32 attached to a curved portion of a pump cassette body outside of a pump chamber 52. The RFID tag 32 is positioned to be aligned with a plunger of an infusion pump when the pump cassette 50 is loaded into the infusion pump. An antenna of an RFID reader can be positioned within a door of the infusion pump for wireless communication with the RFID tag 32 of the pump cassette 50.

FIG. 6 illustrates a pump cassette 60 with an RFID tag 32 attached a pump cassette body facing a splash shield. An antenna of an RFID reader can be positioned behind the splash shield and within a housing of an infusion pump for wirelessly communication with the RFID tag 32 of the pump cassette 60.

FIG. 7 illustrates a pump cassette 70 with an RFID tag 32 attached an outside wall of an air trap 72 of the pump cassette 70. There can be sufficient space to slide the pump cassette 70 into an opening of an infusion pump. An antenna of an RFID reader can be positioned within a door of the infusion pump for wireless communication with the RFID tag 32 of the pump cassette 70. After a door of the infusion pump closes, the RFID tag 32 of the pump cassette can be energized via an antenna of the infusion pump. The antenna of the infusion pump can extend through a shield.

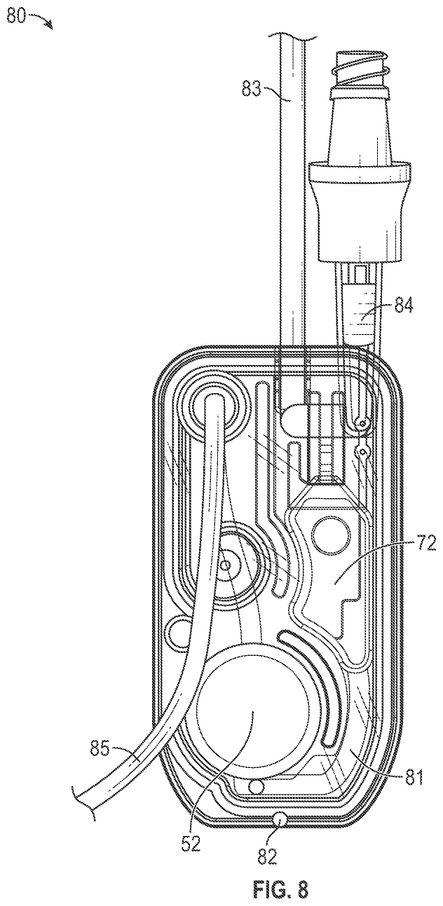

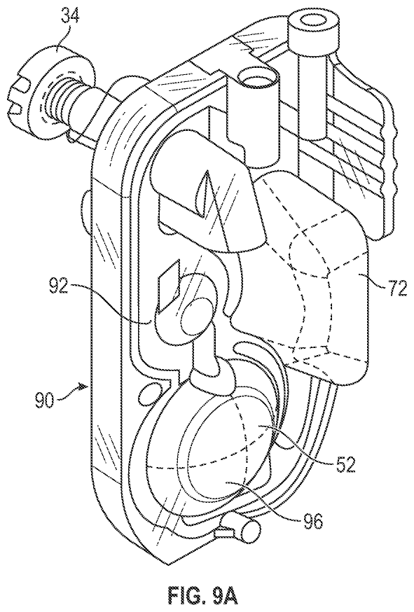

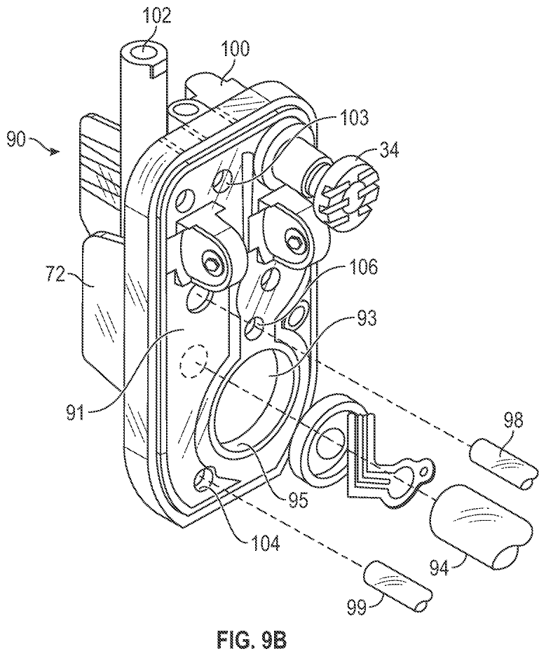

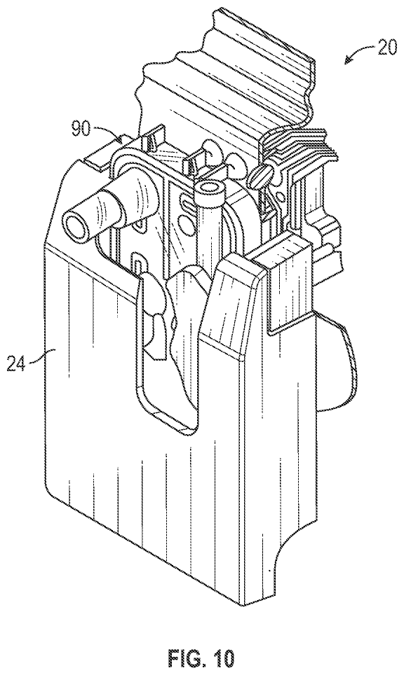

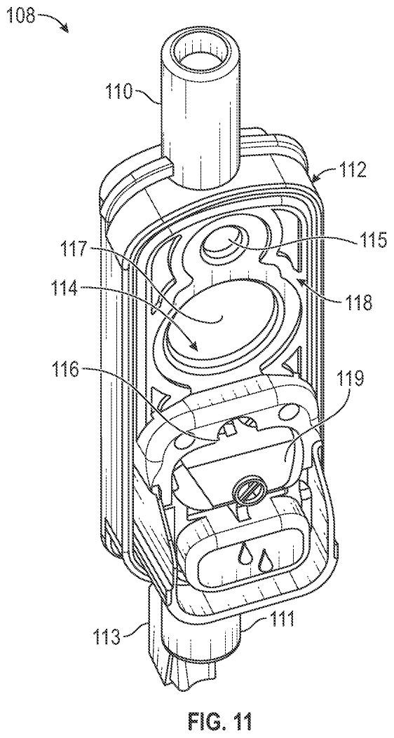

Additional features of pump cassettes that can include an RFID tag will be described with reference to FIGS. 8, 9A, 9B, and 11. A pump cassette can have a housing dimensioned to engage with an infusion pump. FIG. 10 illustrates the pump cassette of FIGS. 9A and 9B loaded into an example infusion pump. Any suitable combination of features of these pump cassettes can be implemented in a pump cassette with an integrated RFID tag. An RFID tag storing any suitable information disclosed herein can be included in and/or implemented with any of the pump cassettes of FIGS. 8, 9A, 9B, and 11. For example, the RFID tag can be embedded in a component, such as a flow stop valve, of any of these pump cassettes. As another example, an RFID tag can be included at any suitable location, such as any of the locations corresponding to the RFID tags of FIGS. 3A-3B, 5, 6, or 7.

FIG. 8 illustrates an example pump cassette 80. The pump cassette 80 includes a U-shaped fluid path 81 within a housing 82. The housing 82 can be referred to as a cassette body. Fluid from a primary line 83 and/or a secondary line connected to a secondary port 84 can flow into the pump cassette 80. The fluid can fill an air trap 72, fill a pumping chamber 52, and then exit the pump cassette 80 through a patient line 85 connected to a patient. The patient line 85 can be referred to as an infusion line.

The pump cassette 80 can be inverted from the illustrated orientation during priming to expel air from the air trap 72 and fill the pumping chamber 52 with fluid. Then the pump cassette 80 can be flipped to the illustrated orientation and loaded into an infusion pump. The pump cassette 80 can receive fluid for infusion from a primary port or a secondary port 84. The secondary port 84 can serve as an attachment point for a secondary infusion. The pump cassette 80 also includes a flow stop value (not illustrated in FIG. 8) on a back of the illustrated side configured to stop fluid flow. In some embodiments, an RFID can be embedded in the flow stop valve.

The infusion pump can control the pump cassette 80 to deliver a set volume of fluid with each pumping cycle. The set volume of fluid delivered during a pumping cycle can be independent of inflow pressure, back pressure, container height, and system resistance. The pumping chamber 52 of the pump cassette 80 can have a flexible silicone diaphragm that is compressed by a piston or plunger of the infusion pump. When the pumping chamber 52 is full and the piston extends, a set volume of fluid is pushed from the pumping chamber 52 into the patient line 85. When the piston retracts, the diaphragm expands and pulls fluid from a container into the pumping chamber 85. The infusion pump can be programmed with a rate that determines the frequency of the piston extension and retraction pumping cycle and thus the volume of fluid delivery.

The pump cassette 80 enables independent control of primary and secondary infusions. The pump cassette 80 can include a valving mechanism that selectively controls fluid flow into the pump cassette 80 for primary and secondary infusions. With the valving mechanism, fluid can flow from the primary port only, the secondary port only, or both the primary and secondary ports. The valving mechanism can enable concurrent fluid delivery of fluid from the primary and secondary ports. For example, the valving mechanism can alternate between drawing fluids into the pump cassette 80 from primary and secondary containers to achieve the programmed administration rate of each fluid.

FIGS. 9A and 9B illustrate an example pump cassette 90. FIG. 9A is a front view of the pump cassette 90 and FIG. 9B is a back view of the pump cassette 90. The pump cassette 90 includes a housing comprising a rigid back member 91 and a rigid face member 92. The pump cassette 90 includes a flow stop valve 34. An elastomeric member 93 is positioned between the back member 91 and the face member 92. The elastomeric member 93 can function as a diaphragm. The back member 91 has plunger opening 95 and the elastomeric member 93 extends across the opening. Behind the plunger opening 95 in the face member 92 is an enlarged recess 96 which forms a pumping chamber 52.

To pump fluid from chamber 52, a plunger 94 of an infusion pump reciprocates into and out of opening 95 urging the diaphragm across opening 95 into and out of the pumping chamber 52. As the plunger 94 of the infusion pump is urged against the diaphragm, a pumping chamber outlet valve actuator 98 is opened while the pumping chamber inlet valve actuator 99 is closed so that fluid is forced from chamber 52 out of a cassette outlet 100. After the plunger 94 delivers a measured amount of fluid from pumping chamber 52, the outlet valve actuator 98 closes, and the inlet valve actuator 99 opens. The plunger 94 is withdrawn from chamber 52 and liquid is drawn into the pumping chamber 52 from a primary cassette inlet 102. The back member 91 also includes valve actuator openings. A primary cassette inlet valve actuator opening 103 is located adjacent primary cassette inlet 102. The inlet actuator opening 103 and the portion of elastomeric member 93 adjacent to the inlet actuator opening 103 form a primary cassette inlet valve.

A pump chamber inlet actuator opening 94 is located upstream of a fluid path leading to the bottom of pump chamber 52. The actuator opening 104 allows a valve actuator 99 to urge a portion of elastomeric member 93 adjacent opening 104 across the fluid path leading into pump chamber 52 to block the flow of fluid into pump chamber 52 from cassette inlet 102 and to block the flow of fluid from pump chamber 52 back through cassette inlet 104. Pump chamber inlet actuator opening 104 and the portion of elastomeric member 93 adjacent opening 104 form a pump chamber inlet valve.

As shown in FIG. 9B, a pump chamber outlet actuator opening 106 is provided through back member 91 to allow pump chamber outlet actuator 98 to urge a portion of elastomeric member 93 to selectively block the flow of fluid from chamber 52. The opening 106 and the portion of elastomeric member 93 adjacent opening 106 form a pump chamber outlet valve.

FIG. 10 illustrates the pump cassette 90 loaded into an infusion pump 20. A portion of infusion pump 20 that includes a door 24 in a closed position is shown in FIG. 6. The pump cassette 90 is engaged with the infusion pump 20 as illustrated.

FIG. 11 illustrates an example pump cassette 108. The pump cassette 108 includes an inlet 110 and an outlet 111 formed in a main body 112. A tube support element 113 is attached to the outlet 111. The tube support element 113 can ensure that that tubing (not shown in FIG. 11) connected to the outlet 111 is maintained in a proper position.

An elastomeric membrane 114 forms an inlet diaphragm 115, an outlet diaphragm 116 (not shown in detail in FIG. 11 but generally indicated as 116), and a pumping chamber 117 located between the inlet and outlet diaphragms 115 and 116, respectively, on an inner face 118 of the main body 111.

In operation, fluid enters through the inlet 110 and is forced through outlet 111 under pressure. The fluid is delivered to the outlet 111 when an infusion pump displaces the pumping chamber 117 to expel the fluid. During an intake stroke, the infusion pump releases the pumping chamber 117, and the fluid is then drawn through the inlet 110 and into the pumping chamber 117. In a pumping stroke, the infusion pump displaces the pumping chamber 117 to force the fluid contained therein through the outlet 111. Accordingly, the fluid can flow through a fluid path of the pump cassette 108 in a series of spaced-apart pulses rather than in a continuous flow. The fluid can be delivered to a patient at a pre-set rate, in a pre-determined manner, and for a particular pre-selected time and/or total dosage.

A flow stop valve 119 is formed as a switch in the main body 112 and protrudes from the inner surface 118. This protrusion forms an irregular portion of the inner surface 118 which can be used to align the pump cassette 108 and/or to monitor the orientation of the pump cassette 108. The flow stop valve 119 can provide a manual switch for closing and opening the pump cassette 108 to fluid flow.

### Infusion Set

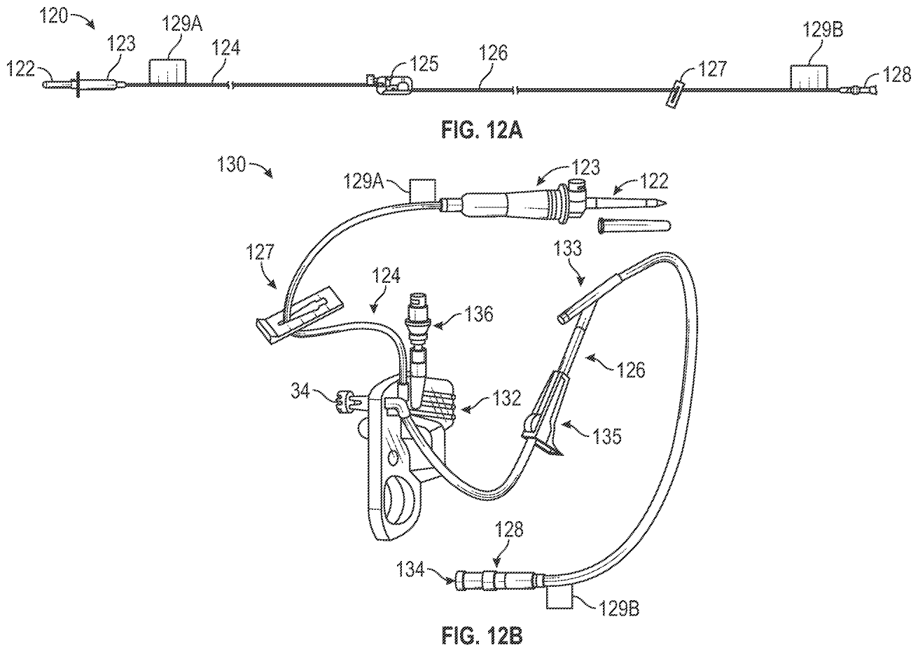

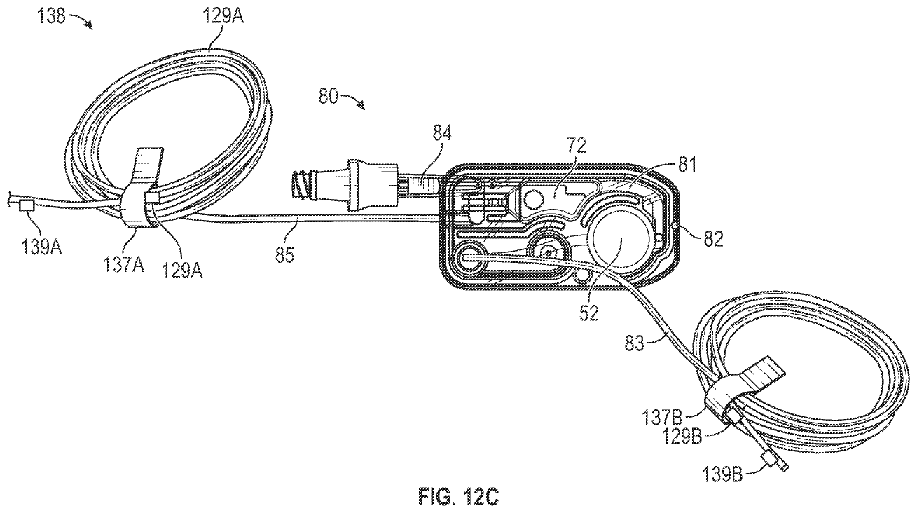

An infusion set can include components for delivering fluid from a fluid source, such as an IV bag or another container, to a delivery device in a patient. The components of the infusion set can be disposable. An infusion set can be provided in a package. An infusion set can include a pump cassette and fluid tubing. The pump cassette can include an RFID tag in accordance with any suitable principles and advantages disclosed herein. Information can be written to the RFID tag during manufacture such that the infusion set is provided with an RFID tag storing information. Any suitable information associated with the infusion set can be stored in the RFID tag of the pump cassette during manufacture. The infusion set can include one or more light sets in accordance with any suitable principles and advantages disclosed herein. The RFID tag of the pump cassette can store pairing information, such as one or more MAC addresses, for wireless communication with the one or more light sets. Accordingly, an infusion pump can automatically initiate a paired wireless communication with the one or more light sets in response to reading information from the RFID tag of the pump cassette. The one or more light sets can be used for line identification, to provide a status indicator and/or an alarm, the like, or any suitable combination thereof. An infusion set together with an infusion pump can provide fluid from a fluid source to a patient. Example infusion sets will be discussed with reference to FIGS. 12A, 12B, and 12C.

FIG. 12A illustrates an example infusion set 120. As illustrated, the infusion set 120 includes a bag spike 122, a drip chamber 123, a primary line 124, a pump cassette 125, a patient line 126, a slide clamp 127, a male luer 128, and light sets 129A and 129B. The bag spike 122 can pierce a container, such as an IV bag. The bag spike 122 is an example of a convertible piercing pin. Infusion fluid for the container can be provided to the pump cassette 125 via the drip chamber 123 and the primary line 124. The primary line 124 includes fluid tubing. The pump cassette 125 can include an RFID tag. The pump cassette 125 has a housing dimensioned to engage with an infusion pump. The infusion fluid can be delivered by the pump cassette 125 to the patient line 126. The patient line 126 includes fluid tubing. The male luer 128 can have a spin collar for secure attachment to a female luer. The light sets 129A and 129B can be attached to and/or integrated with the fluid tubing of the infusion set 120.

FIG. 12B illustrates another example infusion set 130. The infusion set 130 includes a pump cassette 132 connected to a bag spike 122 with drip chamber 123 via a primary line 124 along which a slide clamp 127 can be positioned. The pump cassette 132 can include a RFID tag. In an embodiment, the RFID tag is embedded in a flow stop valve 34 of the pump cassette 132. The pump cassette 132 is also connected to a patient line 126 that includes a Y-site connector 133 along its length and a protective cap with filter 134 at one end. A roller clamp 135 can also be positioned along the length of the patient line 126. The pump cassette 132 in FIG. 12B also includes a secondary inlet port 136 at which a secondary line can be connected. One or more light sets can be attached to and/or integrated with fluid tubing of the infusion set 130. For example, light sets 129A and 129B are illustrated as being attached to the primary line 124 and the patient line 126, respectively.

FIG. 12C illustrates another example infusion set 138. The infusion set 138 includes a pump cassette 80 with a coiled primary line 83 and a coiled patient line 85. A first tape 137A holds the primary line 83 in a coil. Similarly, a second tape 137B holds the patient line 85 in a coil. The first and second tapes 137A and 137B can hold light sets 129A and 129B, respectively, in place. Straightening the lines can remove tape from between a battery and a battery contact of the light sets 129A and/or 129B, for example, as described below. The infusion set 138 is an example of an infusion set that includes one or more RFID tags. The RFID tags 139A and/or 139B can store any suitable information associated with a respective line and/or infusion set. The RFID tags 139A and/or 139B can be attached to respective lines by way of any suitable mechanism, such as tape, attachment ring, hanging tag, or the like. As illustrated, one or more RFID tags 139A and/or 139B can be associated with one or more lines included in and/or attached to the infusion set 138. RFID tags 139A and/or 139A can be included in an infusion set where the pump cassette 80 includes an RFID tag, such as an RFID tag embedded in a flow stop valve. Such an infusion set can also include one or more light sets 129A and/or 129B. In some applications, RFID tags 139A and/or 139A can be included in an infusion set that includes one or more light sets 129A and/or 129B and a pump cassette that does not include an RFID tag.

Line Identification and Light Sets for Line Identification and/or Status Indication

There is a need to identify infusion lines in medical environments, for example, for certain high acuity patients. There can be a plurality of infusion lines connected to such patients. As an example, in an intensive care unit, there can be 6 to 15 infusion lines connected to a single patient at a time. Lines can become tangled and difficult to identify. When an infusion pump alarms (e.g., to indicate an occlusion in a line), it can be difficult for a medical practitioner to locate a particular line associated with the alarm. In some instances, clinicians have been using tape labeling lines at a distal end, a proximal end, and a y-site.

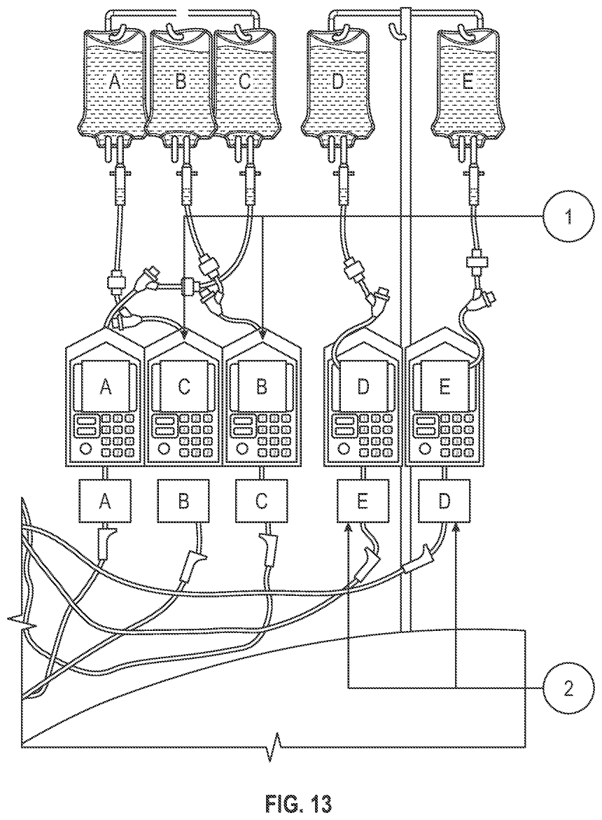

FIG. 13 illustrates an example infusion environment with multiple infusion lines. The multiple infusion lines can connect with a single patient. A plurality of containers are connected to infusion lines via infusion pumps. As shown in FIG. 13, manual labeling can result in errors in properly identifying infusion lines. Technology disclosed herein can reduce and/or eliminate such errors in line identification.

Technical solutions to infusion line identification are provided. Such solutions can be automated. One or more light sets each comprising a light source, such as one or more light emitting diodes (LEDs), can be attached to and/or integrated with medical tuning. Any suitable number of light sets can be attached to and/or integrated with the medical tubing. In certain instances, a first light set can be attached to a proximal end of medical tubing and a second light set can be attached to a distal end of the medical tubing. For instance, a light set can be attached to a tube insertion point. Alternatively or additionally, a light can be attached at a Y-site. As one example, light sets can be located at or near a container, a Y-site, and a catheter, respectively. One or more light sets can be attached to tubing, for example, as described below. Other ways of attaching a light set to tubing include via an attachment ring, hanging tag, or other suitable mechanism. A light set can be attached directly to the medical tubing in certain applications. The light set can be attached to medical tubing so as to inhibit and/or prevent crimping. A light set can be attached to a connector for the medial tubing in some other applications.

An infusion pump can wirelessly communicate with each light set. In embodiments disclosed herein, the infusion pump can determine to illuminate a light set and provide instructions to one or more light sets to illuminate one or more light sources. The infusion pump can send an instruction to illuminate a light source of the light set for line identification. The infusion pump can send an instruction to illuminate a light set to provide an alarm and/or a status indicator. With line identification disclosed herein, infusion lines can be accurately identified without manual labeling. Less manual labeling can be performed for line identification for line identification disclosed herein. Risks associated with erroneous manual labeling can be reduced with line identification solutions disclosed herein.

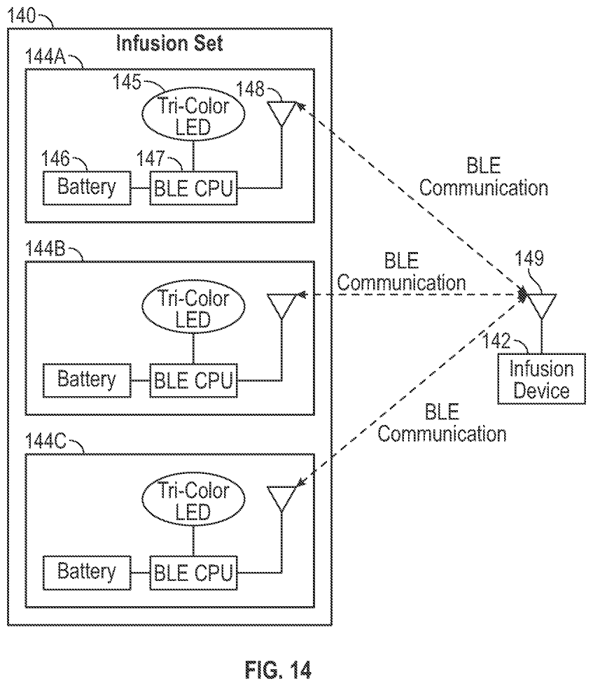

FIG. 14 is a schematic block diagram of an infusion set 140 arranged to wirelessly communicate with an infusion pump 142. The infusion set 140 includes a plurality of light sets 144A, 144B, and 144C. Each light set 144A, 144B, 144C includes a light source 145, a battery 146, a chip 147, and an antenna 148. The light source 145 can include an LED or any other suitable light source. The light source 145 can include a single LED, two or more LEDs of a same color, or two or more LEDs of different colors such as a bicolor LED or tricolor LED. A wireless communication component includes the chip 147 and the antenna 148. The chip 147 can be any suitable chip to facilitate wireless communication. For example, the chip 147 can be a Bluetooth Low Energy (BLE) chip. The antenna 148 can wirelessly transmit and/or receive radio frequency signals. The radio frequency signals can be personal wireless local area network signals, such as BLE signals. The chip 147 can wirelessly exchange information in a power limited environment. The chip 147 can operate in an environment where a wireless link is established with an infusion pump, information is wirelessly exchange, and the link is terminated relatively quickly (e.g., in less than 500 milliseconds). With BLE, the light set can operate in a power limited environment. A wireless connection can be established, used to wirelessly transmit data, and terminated in a relatively short amount of time, such as in less than 500 milliseconds.

Each light set 144A, 144B, 144C can wirelessly communicate with one or more antennas 149 of the infusion pump 142. The infusion pump 142 can control illumination of the light sources 145. The infusion pump 142 can send one or more signals to cause any of the light sets 144A, 144B, 144C to illuminate a respective light source 145. For example, the infusion pump 142 can wirelessly send a command to one or more of the light sets 144A, 144B, 144C light sources 145 to flash an LED for line identification. When the infusion pump 142 alarms, the infusion pump can wirelessly communicate with one or more of the light sets 144A, 144B, 144C to illuminate a respective light source 145. This can indicate that a particular infusion line is occluded or may have another issue.

The light sets 144A, 144B, 144C can be positioned at any suitable locations of an infusion set 140. As an example, light sets 144A, 144B, 144C can be located at or near a container, a Y-site, and a catheter, respectively. Although three light sets 144A, 144B, 144C are sown in FIG. 14, any suitable number of one or more light sets can be included in an infusion set.

Light sets disclosed herein can be electrically inactive until an infusion set is removed from packaging and manually activated. Activation of the light set can be performed without additional actions by a caregiver and/or without additional instructions for the caregiver.

A light set can comprise a housing to protect electronics for a desired use case. The housing can be water resistant and/or waterproof. The light set can be sterilizable via e-beam (e.g., e-beam sterilization at up to 45 kGy). The light set can be compatible with MRI (e.g., MRI at up to 5 T).

A light set can be a standalone device that can be attached to an infusion set at a desired location prior to packaging. The light set can be attached to medical tubing by a method that accommodates for attachment to tubing and/or connectors having a variety of different diameters. The light set can be relatively small and relatively light weight.

The light set can provide illumination that can be visible from all directions and/or consistent with IEC 60601-1-8. The light set can provide at least 96 hours of operation.

There can be a process for pairing MAC addresses for each light set with an infusion pump. For example, a MAC address can be stored in an RFID tag of a pump cassette and the infusion pump can pair with the light set after reading the MAC address from the RFID tag of the pump cassette. The light set can be pre-programmed for wireless communication with an infusion pump that has the MAC address of the light set.

An infusion pump can independently control each light set of an infusion set. Each light set can have a unique address, such as a unique MAC address. Any suitable number of light sets can be included in an infusion set and/or connected to a fluid path of the infusion set. The infusion set can have a direct wireless link with each light set. The infusion pump can cause any of the light sets to illuminate for a particular situation. For example, the infusion pump can cause only a light set connected at or near a distal end of an infusion set to illuminate for a distal occlusion. In response to detecting an alarm condition, the infusion pump can cause a light set connected at or near a proximal end of the infusion set to illuminate, a light set connected at or near a distal end of the infusion set to illuminate, or lights set connected at or near both the proximal and distal ends of the infusion set to illuminate.

The lights sets can include tape to reduce costs and/or improve handling during assembly. The tape can have a rectangular shape and/or a reduced tab end. There can be a variety of different adhesive configurations. For example, there can be relatively simple adhesive patches on a housing with non-adhesive bar code label. As another example, there can be an adhesive on most or an entire bar code label with a deactivated adhesive area and/or a separate release backing to cover adhesive between a battery of the light set and battery contact.

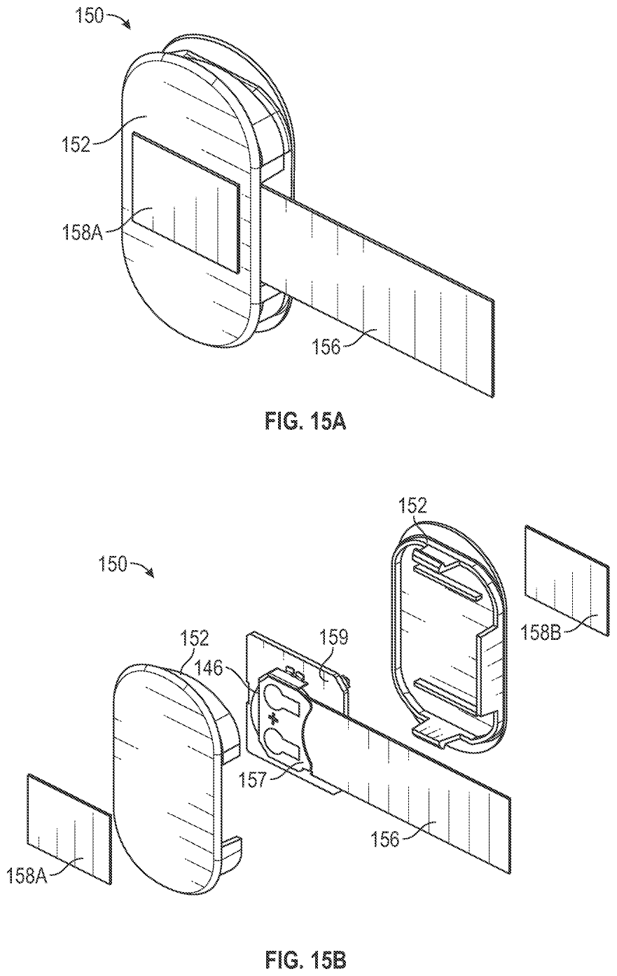

Example light sets will be discussed with reference to FIGS. 15A to 18B. Any suitable combination of features of these light sets can be implemented together with each other. Any suitable combination of features of these light sets can be implemented in any of the infusion sets and/or infusion systems disclosed herein.

FIG. 15A illustrates an assembled light set 150. FIG. 15B illustrates an exploded view of the light set 150. The light set 150 includes a housing 152, a printed circuit board (PCB) module 154, a tape flag 156, and tape assemblies 158A and 158B on opposing out sides of the housing 152. The housing 152 can enclose and protect electronics of the light set 150. The housing 152 can be water resistant and/or waterproof. The PBC assembly 154 includes a PCB 159, a battery 146 on the PCB 159, and a chip 147 (not shown in FIGS. 15A and 15B) on an opposite side of the PCB 159 than the battery 146, and a light source (not shown in FIGS. 15A and 15B). The PCB 159 can be a flexible circuit board. The PCB module 154 can also include an antenna 148 (not shown in FIGS. 15A and 15B).

The tape flag 156 is an insulator tape flag. The tape flag 156 can be a non-adhesive film. The tape flag 156 can have a sequential barcode. As shown in FIG. 15B, the tape flag 156 is positioned between the battery 146 and a battery contact 157 of the PCB 159. The tape flag 156 provides electrical insulation between the battery 146 and the battery contact 157 as shown. The light set 150 can be activated when the tape flag 156 is moved such that the battery 146 is electrically connected to the battery contact 157 of the PCB 159.

Tape assemblies 158A and 158B can each include double sided adhesive tape with a release carrier that is removable to expose an outer adhesive surface. The surface of the tape assembly 158A shown in FIG. 15A has the release carrier over the outer adhesive surface. An adhesive surface of the tape assembly 158B is shown in FIG. 15B. The tape assemblies 158A and 158B can provide tape for a clinician to mark. Accordingly, the clinician can manually mark the light set 150 for any suitable purpose.

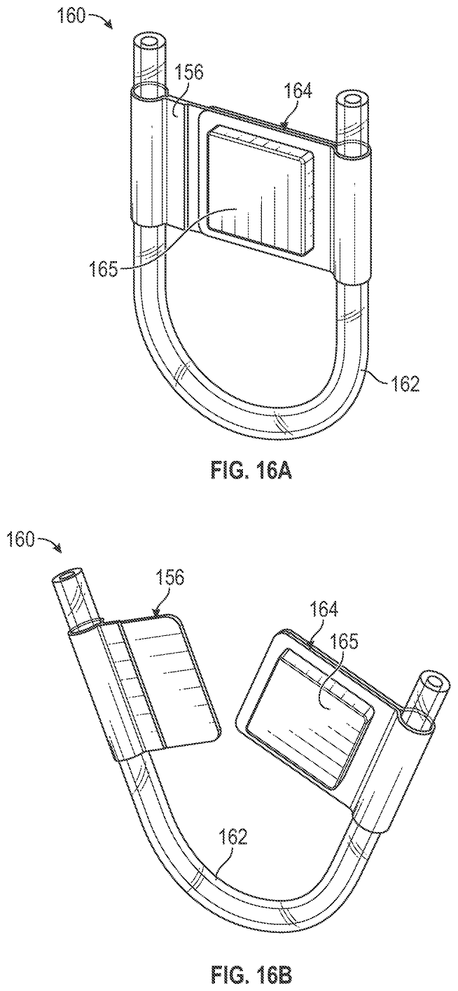

FIG. 16A illustrates a light set 160 attached to tubing 162 before activation of the light set 160. FIG. 16B illustrates the light set 160 after activation. The light set 160 includes a tape 164 that attaches the light set 160 to the tubing 162 and an electronics module 165 having a housing. The tape 164 can have a surface larger than a major surface of the electronics module 165 attached to the tape 164. This can allow for bonding of the electronics module 165 to the tape 164 with strain relief. The tape 164 can have selective adhesive zones to allow the tape flag 156 to be removed, for example, by straightening the tubing 162. In FIG. 16A, the tape flag 156 electrically insulates a battery from a battery contact. Alignment of the selective adhesive zones can be significant such that bonding the tape flag 156 to the tape 164 can be avoided. Features of the electronics module 165 within the housing of the electronics module 165 can be aligned and/or connected concurrent with activation of adhesive of the tape 164.

A user can straighten or otherwise move the tubing 162 such that the tape flag 156 moves to connect a battery within the housing of the electronics module 165 to a battery contact within the electronics module 165. FIG. 16B illustrates the isolator tape flag 156 after the tape flag 156 has been moved from the electronics module 165 and the electronics module 165 is activated.

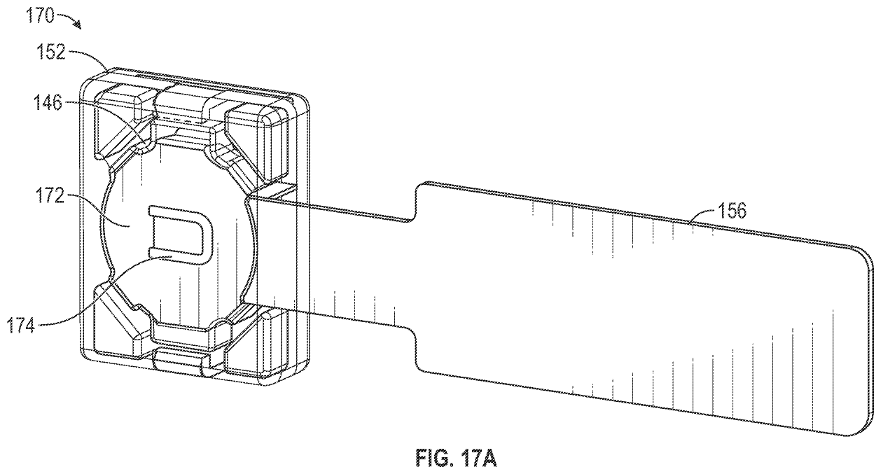

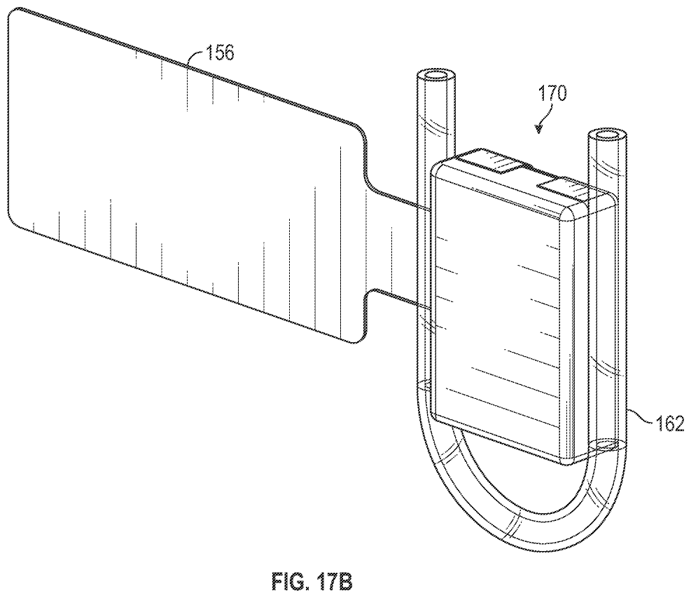

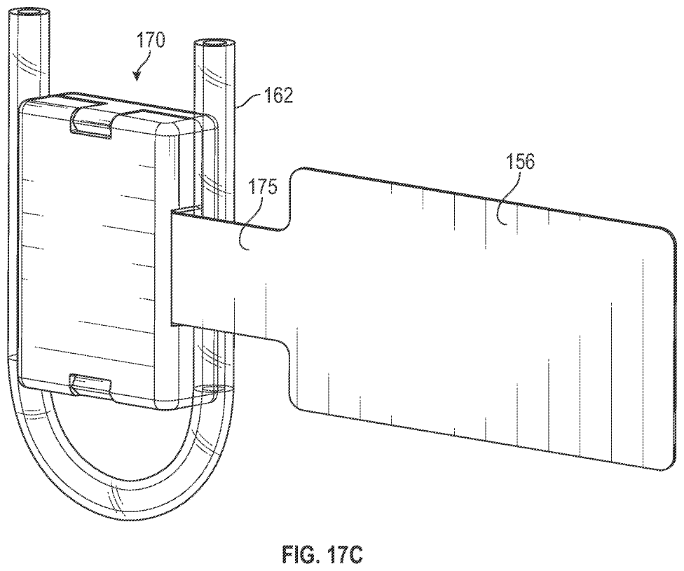

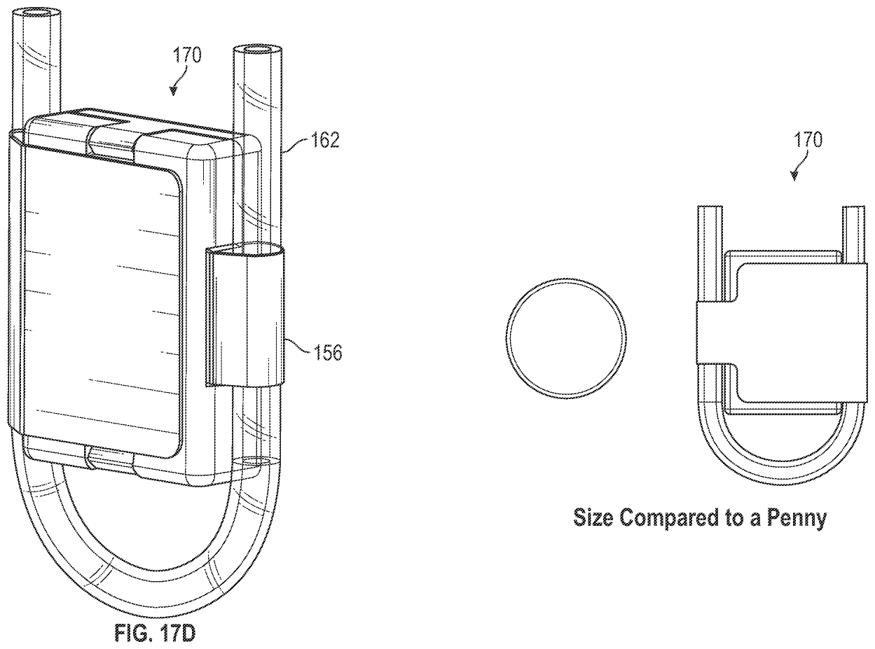

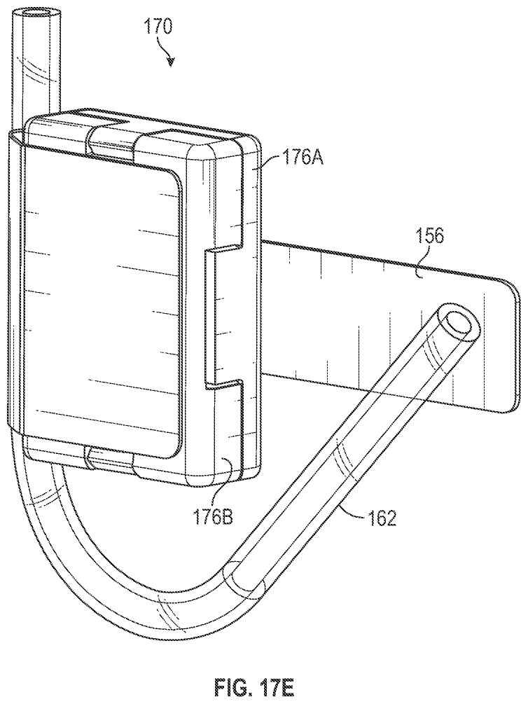

FIGS. 17A to 17E illustrate a light set 170 according to an embodiment. As shown in FIG. 17A, the light set 170 includes a housing 152, a PCB 159 associated with a light source, a battery 146, a battery contact 172 of the PCB 159, and a tape flag 156. A portion 174 of the tape flag 156 is positioned between the battery 146 and the battery contact 172 in FIG. 17A to provide electrical insolation between the battery 146 and the battery contact 172. Accordingly, circuitry of the light set 170 is inactive in FIG. 17A.

FIG. 17B illustrates a light set 170 that can be supplied to a manufacturer with tubing 162. A surface of the tape flag 156 shown in FIG. 17B can have pressure sensitive adhesive. Features of the light set 170 within the housing 152 of the light set 170 can be nested and aligned with the tubing 162 as illustrated. A bottom of the housing 152 in the illustrated orientation can be rounded. A portion of tubing 162 between connectors is shown in FIG. 17B. The tubing 162 can be medical tubing, such as tubing for an infusion line.

FIG. 17C illustrates the reverse side of the light set 170 and fluid tubing 162 relative to FIG. 17B. The tape flag 156 can have printed instructions on a surface shown in FIG. 17C. Tail portions 175 of the tape flag 156 can be free from adhesive on both sides.

FIG. 17D illustrates tape of the tape flag 156 wrapped around fluid tubing 162 and the housing 152. FIG. 17D also illustrates the size of the light set 170 compared to a penny. An infusion set can be provided with one or more light sets 170 attached to fluid tubing 162. The infusion set can be included in a package.

The tape flag 156 can be deployed from between the battery 146 and the battery contact 172 by a user straightening the tubing 162. This can activate the light set 170 by connecting the battery 146 to the battery contact 172. FIG. 17E illustrates the light set 170 attached to tubing 162 after a user has activated the light set 170. FIG. 17E also illustrates locations of a light source on the light set 170. The illustrated light set 170 includes LEDs 176A and 176B. The LEDs 176A and 176C can be illuminated for one or more of line identification, alarming, providing a status indicator, the like, or any suitable combination thereof.

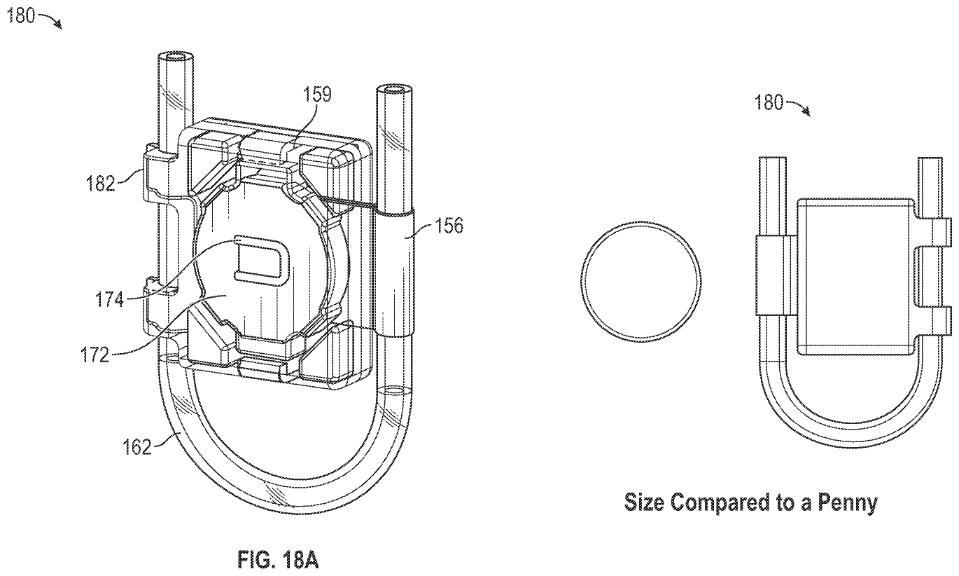

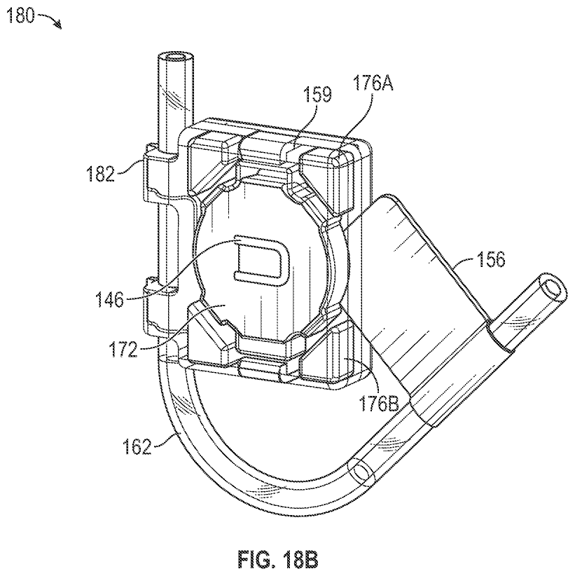

FIGS. 18A and 18B illustrate a light set 180 according to an embodiment. As shown in FIG. 18A, the light set 180 is clamped to tubing 162 and an insulator tape flag 156 connected to the tubing 162 keeps the light set 180 inactive. The light set 180 includes a hinged housing 182 that can connect to the tubing 162. The hinges of the hinged housing 182 and the insulator tape flag 156 can be connected to the tubing 162 on opposite sides of the hinged housing 182. FIG. 18A also illustrates the size of the light set 180 compared to a penny.

The tape flag 156 can be deployed from the hinged housing 182 when the tubing 162 is straightened. The tape flag 156 can be deployed by any other suitable movement of the tubing 162 and/or the light set 180. FIG. 18B illustrates the light set 180 attached to tubing 162 after a user has activated the light set 180. The LEDs 176A and 176C can be illuminated in accordance with any suitable principles and advantages disclosed herein when the light set is activated.

Example use cases for light sets will now be described. Any of these use cases can be implemented in association with any suitable light set in accordance with any suitable principles and advantages disclosed herein.

A use case related to establishing a wireless connection between a light set and an infusion pump will be described. A user can configure the infusion pump. The user can be a nurse, for example. The infusion pump can start transmitting advertising packets. A user can press a button on the light set or the light set can otherwise be activated, for example, by moving a tape flag from between a battery and a battery contact. The light set can send a connection request. The infusion pump can request an authenticate key, which can be a set identifier (e.g., a set serial number) which can already be stored in the light set. In some other applications, the authentication key can be obtained from an RFID tag of a pump cassette. A connection can be established upon successful authentication. In certain applications, one color LED (e.g., a green LED) can be illuminated in response to a successful authentication and another color LED (e.g., a red LED) can be illuminated in response to unsuccessful authentication. The light source can send a heartbeat to the infusion pump in response to the connection being established.

After a wireless connection is established between the light set and the infusion pump, the light set can send battery status information to the infusion pump. If the battery status satisfies a threshold indicating sufficient battery life, the wireless connection will remain established and a light source (e.g., a green LED) can be illuminated for a specific time. Then a clinician can begin and/or continue infusion. On the other hand, if the infusion pump determines that the battery status does not satisfy the threshold, the light set can turn off and provide a different illumination of the light source (e.g., illuminate a different light pattern and/or illuminate a different color LED such as a red LED). This can indicate to the battery is running low and should be replaced. The infusion pump can also indicate that the battery level of the light set battery is low. A clinician can take action and replace the battery.

The light set can indicate pump status. The infusion pump can control the light set to indicate the pump status. After a wireless connection is established and the infusion is running, the light set can send heartbeat indicating the wireless connection remains established and the light set is listening for events. If the infusion is running as planned, the infusion pump can send a command to illuminate a light source of the light set a certain way. For example, the light source can be illuminated with a particular color of light (e.g., green) and/or a particular pattern (e.g., continuous illumination, blinking at a particular interval, and/or illumination for a particular length of time). In response to detecting a pause in infusion, the infusion pump can send a command to illuminate the light source of the light set to indicate the pause. For example, the light set can be illuminated with a different color of light (e.g., yellow) and/or a different pattern than for infusion running as planned. The infusion pump can send a command to illuminate the light source of the light set to indicate an issue with infusion, such as infusion stopping and/or an occlusion. To indicate the issue with infusion, the light source of the light set can be illuminated differently than for infusion proceeding as planned and/or being paused. As an example, the light source can blink red light. The infusion pump can cause the light source to be illuminated differently for manual stopping compared to an alarm. For instance, manual stopping can be indicated by a single blink of light and an alarm can be indicated by continuous blinking for a longer period of time.

The light set can indicate that a wireless link between the light set and the infusion pump is terminated. In response to infusion being complete, the infusion pump can cause the light source of the light set to indicate infusion is complete. As an example, the light set can blink yellow light to indicate infusion is complete. In response to not receiving a communication from the infusion pump for a certain period of time, the light source of the light set can indicate that the wireless connection is being terminated. In response to a determination that the battery is at a low level, the wireless link between the light set and the infusion pump can be terminated and the light source can be illuminated to indicate a low battery level. In certain instances, a low battery level warning indicator can be provided by the infusion pump before terminating the wireless link and/or turning off the light set.

An infusion pump can illuminate light sets of an infusion set. The pump can determine a status associated with infusion, such as normal operation, infusion paused, an issue with infusion (e.g., infusion being stopped, an occlusion, a pump cassette and/or infusion set timing out, a pump cassette and/or infusion set exceeding use parameters, a potentially unsafe condition associated with infusion, etc.), or infusion being terminated. The infusion pump can wirelessly transmit a first instruction to a plurality of light sets of the infusion set that are connected to a fluid pathway of the infusion set. The first instruction can cause a respective light source of each of the light sets to illuminate. The infusion pump can wirelessly transmit a second instruction to at least one light set of the plurality of light sets. The second instruction can cause the light source of the at least one light set to illuminate differently than the first instructions to indicate a different status associated with infusion.

The second instruction can cause the light source to provide a different color of light than the first instruction. The second instruction can cause the light source to provide light for a different duration of time than the first instruction. The second instruction can cause the light source to provide flashes of light at a different interval than the first instruction. The second instruction can be associated with an issue with infusion.

There can be a direct wireless link between the infusion pump and each of the light sets. The infusion pump can control each light set independently.

### Other Considerations

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. The word “coupled”, as generally used herein, refers to two or more elements that may be either directly coupled, or coupled by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. A pump cassette for fluid delivery, the pump cassette comprising:
a housing dimensioned to engage with an infusion pump;
a fluid path within the housing, the fluid path configured to receive fluid from a fluid source and to deliver the fluid to tubing of an infusion line; and
a radio frequency identification tag embedded in a component of the pump cassette.

2. The pump cassette of claim 1, wherein the radio frequency identification tag is surrounded by molding material of the component.

3. The pump cassette of claim 1, wherein the component is a mechanical component.

4. The pump cassette of claim 1, wherein the component is a flow stop valve.

5. The pump cassette of claim 1, wherein the radio frequency identification tag stores an identifier uniquely identifying the pump cassette.

6. The pump cassette of claim 1, wherein the radio frequency identification tag stores pairing information for wireless communication with a light set.

7. The pump cassette of claim 1, wherein the radio frequency identification tag stores cassette usage information.

8. The pump cassette of claim 1, wherein the radio frequency identification tag stores information identifying one or more pump cassette parameters.

9. The pump cassette of claim 1, wherein the fluid path comprises an air trap and a pumping chamber.

10. The pump cassette of claim 1, wherein the pump cassette is configured to independently control pumping for a primary infusion and a secondary infusion.

11. The pump cassette of claim 1, wherein the fluid path is U-shaped.

12. The pump cassette of claim 1, wherein the pump cassette is configured to deliver a set volume of the fluid with each pumping cycle.

13. An infusion pump for fluid delivery, the infusion pump comprising:
a housing configured to a receive a pump cassette; and
an antenna configured to wirelessly communicate with a radio frequency identification tag of the pump cassette when the pump cassette is engaged with the housing, wherein the antenna is arranged to be aligned with a flow stop valve of the pump cassette when the pump cassette is engaged with the housing,
wherein the infusion pump is configured to control infusion of fluid to a patient.

14. The infusion pump of claim 13, further comprising a processor configured to perform a safety check based on information from the radio frequency identification tag read by way of the antenna, and to cause infusion of the fluid based on passing the safety check.

15. A method of operating an infusion pump, the method comprising:
reading information from a radio frequency identification tag embedded in a component of a pump cassette; and
controlling infusion, by the infusion pump, based on the information from the radio frequency tag.

16. The method of claim 15, further comprising writing, by the infusion pump, to the radio frequency identification tag during infusion.

17. The method of claim 15, further comprising writing information indicative of a start time of infusion to the radio frequency identification tag.

18. The method of claim 15, further comprising writing information indicative of a medication delivered to a patient to the radio frequency identification tag.

19. The method of claim 15, wherein the controlling infusion comprises verifying a pump cassette based on the information.

20. The method of claim 15, wherein the controlling infusion comprises performing a safety check related to at least one of previous usage of the pump cassette or a condition of the pump cassette.
